(19) United States
(12) Patent Application Publication
(10) Pub. No.: US 2009/0318678 A1
(43) Pub. Date: Dec. 24, 2009

(54) Stable C-Glycoside Sugar and C-Glycoconjugate Mimetics, Method for preparing same and uses Thereof in Particular in Cosmetics and Drugs

(75) Inventors: Géraldine Castelot-Deliencourt-Godefroy, Rouen (FR); Jean-Charles Quirion, Bourg-Achard (FR); Philippe Jubault, Preaux (FR); Ludivine Zoute, Sailly Lez Lannoy (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: TFCHEM, Rouen (FR)

(21) Appl. No.: 12/226,586

(22) PCT Filed: Apr. 23, 2007
(86) PCT No.: PCT/FR2007/000683

§ 371 Date: Apr. 3, 2009

(30) Foreign Application Priority Data
Apr. 27, 2006 (FR) 06/03823

(51) Int. Cl. C07H 7/00

(52) U.S. Cl. 536/53; 536/115; 536/122

(57) ABSTRACT

The invention concerns a C-glycoside compound of formula (I); wherein: n is equal to 1 or 2; Y represents H or halogen; X is an alkyl chain bearing at least one amino, amide, acid, ester, carbonyl, alcohol, aryl function or a carbonyl, ester amide, amino, alcohol group; the R's, identical or different, represent a OH or OR′ group where R′ is an alkyl, benzyl, benzoyl, acetyl, pivaloyl, trialkylsilyl, tertiobutyldiphenylsilyl group or one or more sugars; R1 represents OR′, NR″R″′, N3, or a phthalamide with R″ and R″′, identical or different, represent H or an alkyl, aryl, benzyl, benzoyl, acetyl, alkoxycarbonyl, allyloxycarbonyl, benzyloxycarbonyl group; R2 represents H or halogen or a OH, OR, NR″R″′ or N3 group, as well as derivatives thereof in physiologically or pharmaceutically acceptable base, mineral or organic acid-addition salt, hydrate or solvate form. The invention is useful for preparing C-glycoside compounds or C-glycoconjugates applicable in particular in cosmetology, medical imagery, immunology for treating cancer, diabetes, hypertension.

The present invention relates to a novel family of C-glycoside and C-glycoconjugate sugar mimetics useful in a number of fields such as cosmetics and medical imaging, as well as in pharmaceutical applications such as, for example, as an antifungal, antiparasitic, antithrombotic, antibiotic, antiviral, anti-infective, anti-inflammatory, antipsychotic, antidepressant or antineoplastic.

Generally, sugars are known to constitute a fundamental class of biomolecules involved in a variety of functions: In addition to constituting forms of energy reserves, they participate in cellular communications and immune system functioning, they help the organism fight against pathogenic microorganisms, and they intervene in the process of cancerization. It is thus this ability to communicate with other cells, proteins, hormones, viruses, toxins and bacteria that makes sugars a veritable arsenal for developing novel treatments, most notably in the area of cancers, viruses, inflammation and many others.

Sugars are present in known drugs such as drugs of the cardiovascular system with cardiac glycosides, anticoagulants with heparin, aminoglycoside or glycopeptide antibiotics, cytotoxic antibiotic antineoplastics, etc. Moreover, adding water-soluble sugars to a drug's active ingredients improves their solubility in biological media and modifies their pharmacokinetic properties (circulation, elimination and concentration in biological media). Glycosylation can also delay the break-down process (this is the case in particular with opioid peptides such as enkephalins) and influence transport across a number of barriers such as the blood-brain barrier, thus blocking entry in the brain or facilitating transport by targeting active glucose transport systems. Moreover, glycosylation can also strengthen interactions with receptors or lectins present on the cell surface and thus induce greater vectorization and selectivity in the form of glycoconjugates.

Unfortunately, in spite of the therapeutic potential of glycosides and their derivatives, their commercial use for developing novel drugs is quite often impeded by significant disadvantages:
high cost and difficulties with synthesis, purification and analysis,
high instability during chemical and enzymatic hydrolysis processes and thus rapid break-down in the organism, most notably by glycosidases present in large numbers in this medium.

Appreciation of the therapeutic potential and limitations of sugars has fueled the search for mimetics of these structures for use as novel access routes for more effective therapeutic agents. Although some of these discoveries have applications, they tend to be limited. Thus a wide variety of hybrid sugar structures have been developed. Among these, C-glycosides constitute a major advance in resolving problems of stability with sugars and their derivatives.

In this area, two families have emerged, CH₂— and CF₂-glycosides, in which the anomeric oxygen is replaced by a CH₂ or CF₂ group in order to eliminate the problem of sugar stability. However, although CH₂ is highly advantageous in terms of stability, replacing the anomeric oxygen with this group causes significant changes in terms of electronegativity, polarity and thus the behavior of the novel sugar in biological phenomena. Replacing the anomeric oxygen with a CF₂ leads to good stability, and especially to excellent substitution in terms of electronegativity. However, the presence of CF₂, due to the inductive attractor effect of the two fluorine atoms, can sensitize nearby functions such as carbonyls which can then be attacked by nucleophilic functions such as amines.

More particularly, the aim of the invention is to eliminate these disadvantages with a novel family of sugar mimetics, C-glycosides and C-glycoconjugates, in which the oxygen of the anomeric function is replaced by a group comprising a carbon atom carrying a fluorine atom, stable under enzymatic break-down and acid-base hydrolysis, and exhibiting reduced sensitivity in the face of nucleophilic attacks.

More particularly, the invention provides a stabilized C-glycoside compound which when used as an analog or adduct/vehicle for biologically active compounds can improve their activity.

According to the invention, this C-glycoside compound has the following formula (I):

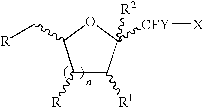

wherein:
n is an integer equal to 1 or 2,
Y represents an atom of hydrogen, chlorine or bromine,
X is an atom of hydrogen or a linear or branched alkyl chain with at least one amine, amide, acid, ester, carbonyl, alcohol or aryl function or a carbonyl, ester, amide, amine or free or protected alcohol group,
R units are identical or different and represent an OH or OR′ group,
wherein R′ is a linear or branched alkyl, benzyl, benzoyl, acetyl, pivaloyl, trialkylsilyl, tertiobutyldiphenylsilyl group or one or more sugars,
R¹ represents OR′, NR″R″′, N₃, or a phthalimide,
   R″ and R″′, identical or different, represent an atom of hydrogen or a linear or branched alkyl, aryl, benzyl, benzoyl, acetyl, alkyloxycarbonyl, allyloxycarbonyl or benzyloxycarbonyl group,
R² represents an atom of hydrogen or a halogen, preferably a halogen chosen among F, Cl, Br or I, or an OH, OR, NR″R″′ or N₃ group,
   as well as derivatives of same in the form of a base, a mineral or organic acid addition salt, a hydrate or a physiologically or pharmaceutically acceptable solvate.

The linear or branched alkyl groups can be groups having one to 15 carbon atoms.

This novel family of monofluorinated C-glycosides and C-glycoconjugates advantageously provides:
1. Glycoconjugate stabilization: analog preparations modified by this glycoside and glycoconjugate technology will have improved stability, bioavailability and thus effectiveness compared to their parent compounds. Moreover, they can be more easily administered by oral route. This will improve their use as a drug.
2. Neoglycosylation: preparation of glycosylated versions of drug active ingredients. Adding water-soluble sugars to drug active ingredients improves their solubility in biological media and modifies their pharmacokinetic properties (circulation, elimination and concentration in biological media). Glycosylation can also delay break-down processes (this is the case in particular with peptides such as opioid peptides: enkephalins), influence transport across a number of barriers such as the blood-brain barrier, and thus block entry in the brain or facilitate transport by targeting active glucose transport systems. Glycosylation is also important for barriers such as the placental barrier and can thus prevent fetal intoxication. Moreover, glycosylation can also strengthen interactions with receptors or lectins present on the cell surface and thus induce greater vectorization and selectivity in the form of glycoconjugates. In addition, these compounds benefit from greater stability and from the effect of introducing the fluorine atom.

The invention also relates to a method for preparing compounds of formula (I).

According to a first embodiment, said compounds of formula (I) wherein Y represents a hydrogen molecule can be obtained by a method comprising the reaction of an alkyl dibromofluoroacetate in the presence of diethylzinc and triphenylphosphine with lactones of formula (II):

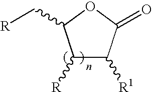

with n, R and R¹ as previously defined.

Compounds of formula (I) wherein X and Y are hydrogen atoms and R² represents a hydroxyl (OH) can be obtained as secondary products of the reaction of a compound of formula (I) wherein R²═OH, Y═H and X═CO₂H in the presence of a peptide coupling agent such as 3-ethyl-1(N,N-dimethylaminopropyl)carbodiimide (EDCI) or dicyclohexyl carbodiimide (DCC) in the presence of a tertiary amine such as N-methylmorpholine (NMM) or diisopropylethylamine (DIEA).

According to a second embodiment, said compounds of formula (I) wherein Y represents a hydrogen molecule can be obtained by a method comprising a Reformatsky addition reaction of an alkyl bromofluoroacetate in the presence of zinc with lactones of formula (II).

Advantageously, the choice of one or the other of these two embodiments favors one or the other of the configurations of the asymmetric center carrying the fluorine atom.

According to a third embodiment, said compounds of formula (I) wherein Y represents a halogen atom such as chlorine or bromine can be obtained by a method comprising a reaction of alkyl dihalofluoroacetate in the presence of diethylzinc with lactones of formula (II).

Said lactones can be obtained by traditional steps of protection by benzylation of sugar, followed by acid hydrolysis of the anomeric position and then its oxidation.

Compounds of general structure (I) with R═OH can be halogenated to obtain compounds of general structure (I) with R²═Cl or Br and then reduced to obtain compounds of general structure (I) with R²═H.

Compounds of formula (III), also obtained by the preparation method using diethylzinc, dibromofluoroacetate and triphenylphosphine, can constitute active compounds:

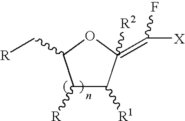

with n, R, R¹ and X as previously defined.

More specifically, compounds of formula (III) wherein X═Br can be obtained by reacting the lactone of formula (II) in the presence of tribromofluoromethane (CFBr₃), triphenylphosphine and diethylzinc.

Compounds of formula (III) wherein X═H can be obtained by reacting a compound of formula (I) wherein X═CO H, R²═OH and Y═H in the presence of a peptide coupling agent such as 3-ethyl-1(N,N-dimethylaminopropylcarbodiimide (EDCI) or dicyclohexyl carbodiimide (DCC) in the presence of a tertiary amine such as N-methylmorpholine (NMM) or diisopropylethylamine (DIEA).

In compounds of general formula (III), the double bond can be reduced to yield compounds of general formula (I) with R²═H and Y═H, but also:

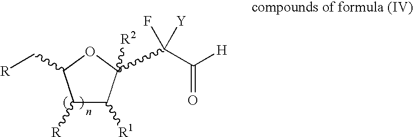

wherein:
n is an integer equal to 2,
Y represents an atom of hydrogen,
R² represents an atom of hydrogen or an OH or OR group,
and obtained from the ester (X═CO₂Et in formula (I)), either by reduction with diisobutylaluminum hydride (DIBALH), or by reduction to an alcohol (formula (V)) followed by oxidation;

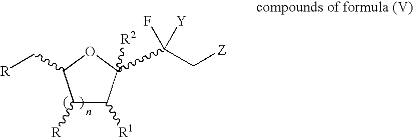

wherein:
n is an integer equal to 2,
Y represents an atom of hydrogen,
R² represents an atom of hydrogen or an OH or OR group,
Z represents OH or OR³ with R³=alkyl, benzyl, mesyl, tosyl, triflate or a halogen such as Cl, Br or I;

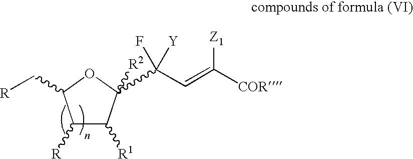

wherein:
n is an integer equal to 2,
Y represents an atom of hydrogen,
R² represents an atom of hydrogen or an OH or OR group,
Z₁ represents H or NR″R″′ with R″ and R″′, identical or different, representing an atom of hydrogen or a linear or branched alkyl, aryl, benzyl, benzoyl, acetyl, alkyloxycarbonyl, allyloxycarbonyl or benzyloxycarbonyl group,
R″″ represents OR″ or NR″R″′ or an amino acid obtained by Wittig-Wadsworth-Horner-Emmons reaction of a phosphonate with the aldehyde of formula (IV);

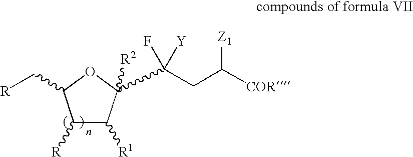

wherein:
n is an integer equal to 2,
Y represents an atom of hydrogen,
R² represents an atom of hydrogen or an OH or OR group,
Z₁ represents H or NR″R″′ with R″ and R″′, identical or different, representing an atom of hydrogen or a linear or branched alkyl, aryl, benzyl, benzoyl, acetyl, alkyloxycarbonyl, allyloxycarbonyl or benzyloxycarbonyl group,
R″″ represents OR″ or NR″R″′ or an amino acid obtained by reducing the double bond of formula (VI);

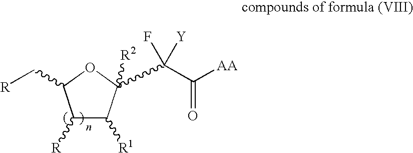

wherein:
n is an integer equal to 2,
Y represents an atom of hydrogen,
R² represents an atom of hydrogen or an OH or OR group,
   and obtained from the ester (X═CO₂Et in formula (I)) by saponification to yield the acid (X═CO₂H in formula (I)) by the action of lithium oxide or soda or potash, followed by peptide coupling with an amino acid (AA) or a peptide, i.e., linking of amino acids in the presence of traditional coupling agents such as dicyclohexyl carbodiimide (DCC), 3-ethyl-1(N,N-dimethylaminopropyl carbodiimide (EDCI), (2-(7-Aza-1H-benzotriazole-1-yl)-1,1,3,3-tetramethyluronium hexafluorophosphate) (HATU), (2-1H-benzotriazole-1-yl)-1,1,3,3-tetramethyluronium hexafluorophosphate (HBTU) with or without hydroxybenzotriazole (HOBt), and a tertiary amine such as N-methylmorpholine (NMM) or diisopropylethylamine (DIEA) or triethylamine (Et₃N);

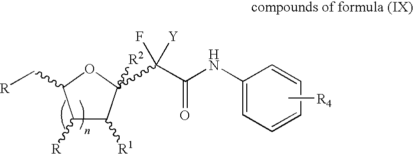

wherein:
n is an integer equal to 2,
Y represents an atom of hydrogen,
R² represents an atom of hydrogen or an OH or OR group,
R⁴ represents a hydrogen, halogen, NR″R″′, OH or OR″,
with R″ and R″′, identical or different, representing an atom of hydrogen or a linear or branched alkyl, aryl, benzyl, benzoyl, acetyl, alkyloxycarbonyl, allyloxycarbonyl, benzyloxycarbonyl, or obtained from the ester (X═CO₂Et in formula (I)) by saponification to yield the acid (X═CO₂H in formula (I)) by the action of lithium oxide or soda or potash, followed by coupling with an aromatic amine in the presence of traditional coupling agents such as DCC, EDCI, HATU, HBTU, with or without HOBt, and a tertiary amine such as NMM, DIEA or Et₃N.

The invention also relates to a C-glycoside compound of formula (I) wherein radical R² consists of an OH group present, when it is in solution in polar and protic solvents, in the various traditional forms of sugars in solution, namely the open forms furanose and pyranose.

Thus, for example, in galactose series, the compound will have following formula (X):

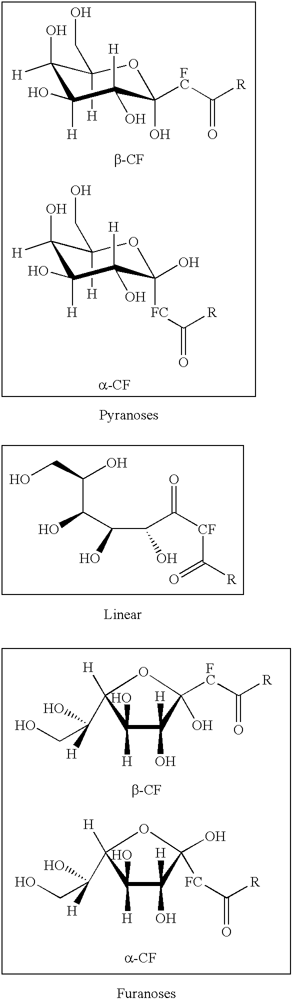

The advantage of the C-glycoside or C-glycoconjugate compounds according to the invention compared to prior technologies and to natural glycosides and glycoconjugates resides in:
The existence of electronegativity due to the presence of a fluorine atom preserves the molecule's polarity.
Its particular resistance to enzymatic break-down and to acid-base hydrolysis yields non-hydrolyzable compounds (chemically and enzymatically), which has a beneficial effect on bioavailability and half-life when these compounds are used as drugs. It thus improves metabolic stability by blocking a metabolic site with a fluorine atom, an atom sufficiently small not to interfere with receptor interactions.
An inductive effect of the fluorine atom, which does not have a fragilization effect on neighboring functions when encountering nucleophiles.
The introduction of an asymmetric center at the anomeric position will lead to improved affinity in receptor interactions because this position is often involved in natural glycoside receptor interactions.
Changes in physical/chemical properties, most notably with respect to the acidity and alkalinity of neighboring functions, are highly similar in the case of the introduction of a single fluorine atom to compounds having an oxygen. Often, changes in pKa have a strong effect on a molecule's pharmacokinetic properties and interactions.
The impact on interactions with proteins can be a direct effect between the fluorine and the protein or an indirect effect via functions near the fluorine whose polarity is thus modified, given that C—F . . . C═O interactions can play an important role by thus significantly increasing interactions.

Non-limiting examples of preparation of the compounds according to the invention are described below and refer to the annexed illustrations wherein:

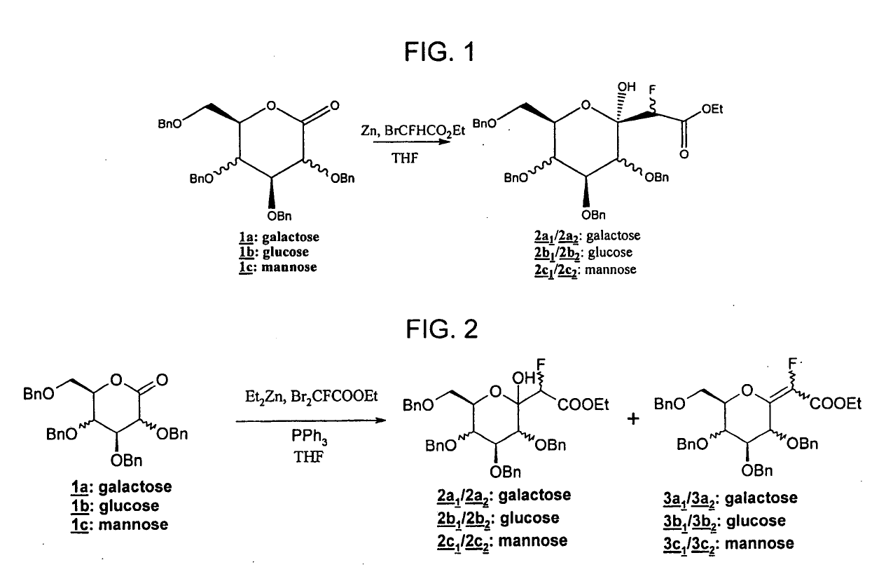

FIG. 1 is a reaction equation for obtaining compound 2a₁/2a₂; 2b₁/2b₂; 2c₁/2c₂;

FIG. 2 is a reaction equation for obtaining compound 2a₁/2a₂; 2b₁/2b₂; 2c₁/2c₂; as well as 3a₁/3a₂; 3b₁/3b₂; 3c₁/3c₂;

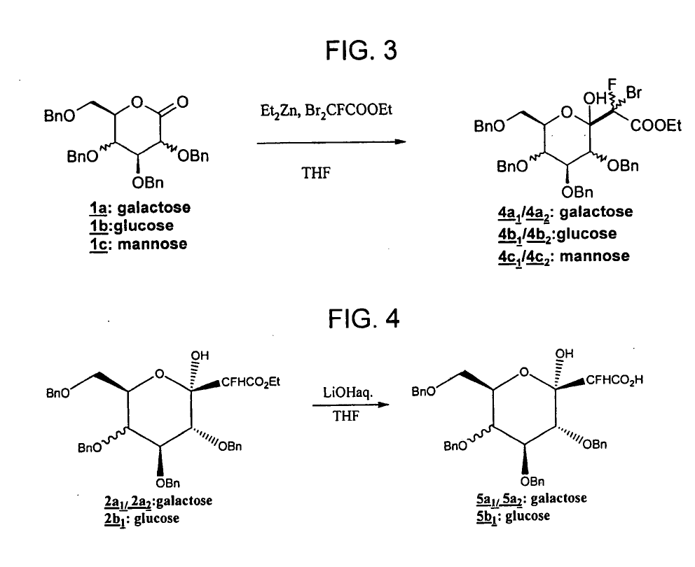

FIG. 3 is a reaction equation for obtaining compound 4a₁/4a₂; 4b₁/4b₂; 4c₁/4c₂;

FIG. 4 is a reaction equation for obtaining compound 5a₁/5a₂; 5b₁;

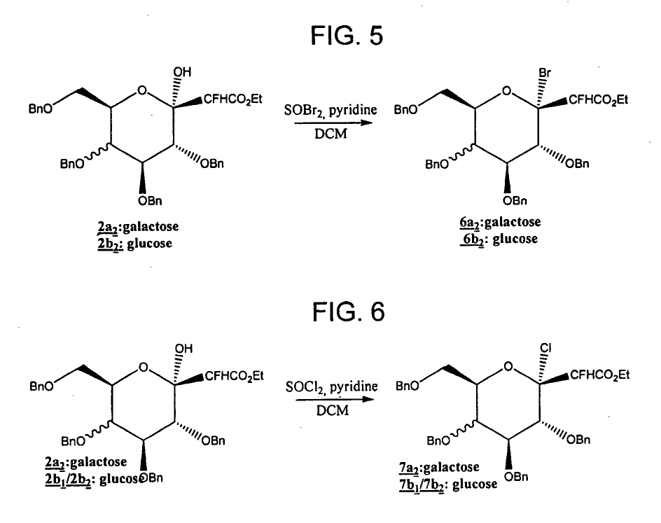

FIG. 5 is a reaction equation for obtaining compound 6a₂; 6b₂;

FIG. 6 is a reaction equation for obtaining compound 7a₂; 7b₁/7b₂;

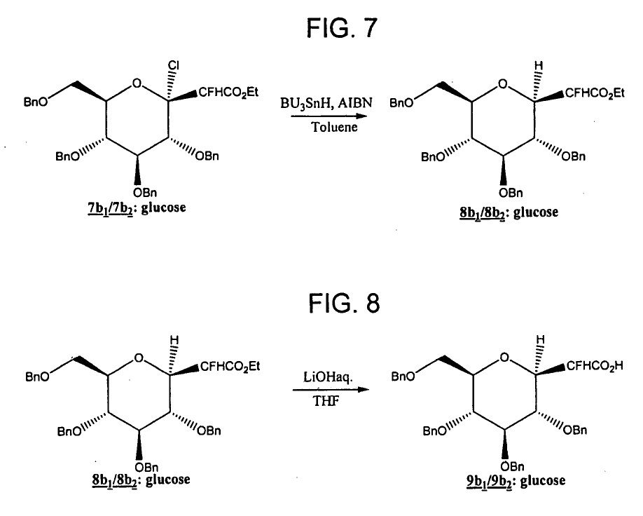

FIG. 7 is a reaction equation for obtaining compound 8b₁/8b₂;

FIG. 8 is a reaction equation for obtaining compound 9b₁/9b₂;

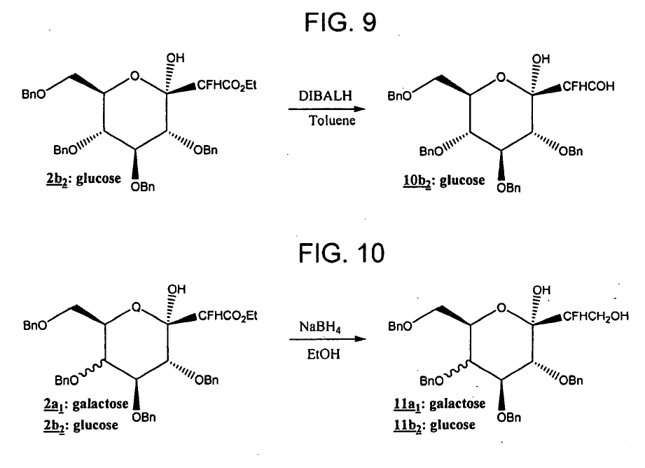

FIG. 9 is a reaction equation for obtaining compound 10b₂;

FIG. 10 is a reaction equation for obtaining compound 11a₁; 11b₂;

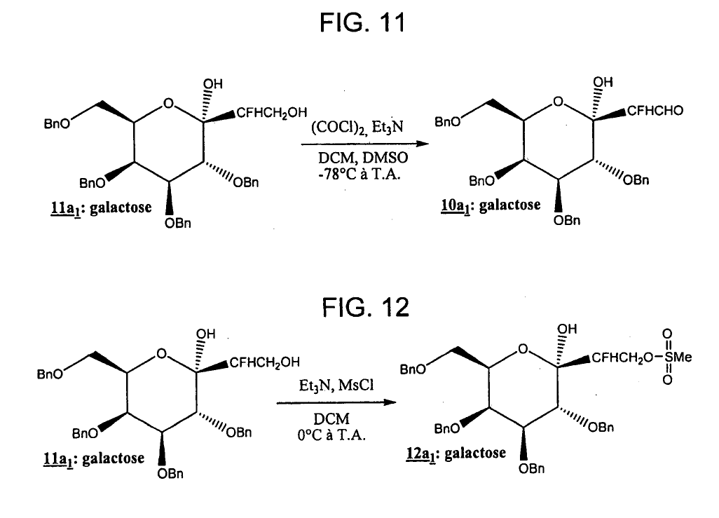

FIG. 11 is a reaction equation for obtaining compound 10a₁;

FIG. 12 is a reaction equation for obtaining compound 12a₁;

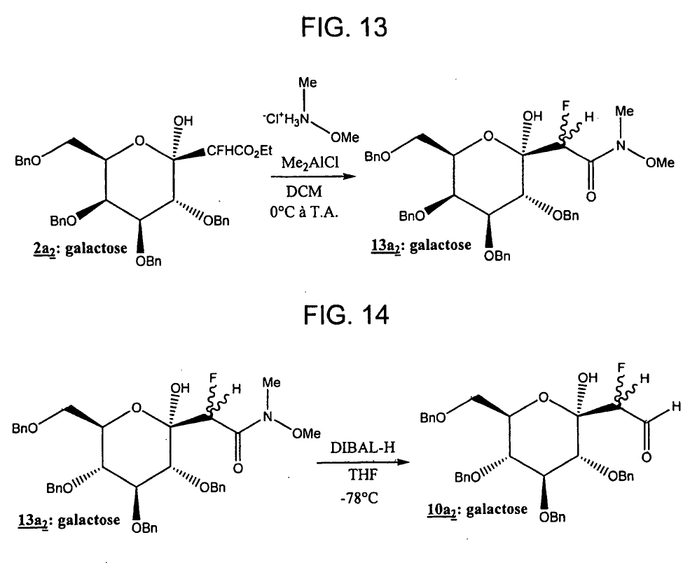

FIG. 13 is a reaction equation for obtaining compound 13a₂;

FIG. 14 is a reaction equation for obtaining compound 10a₂;

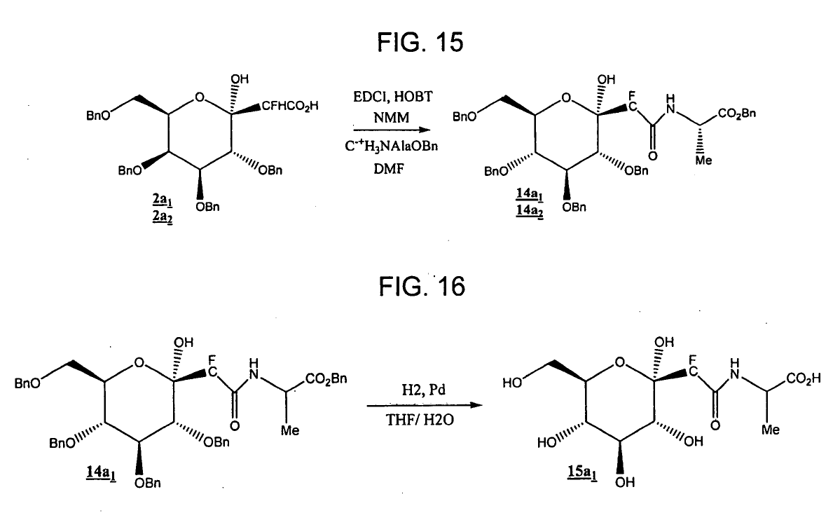

FIG. 15 is a reaction equation for obtaining compound 14a₁/14a₂;

FIG. 16 is a reaction equation for obtaining compound 15a₁;

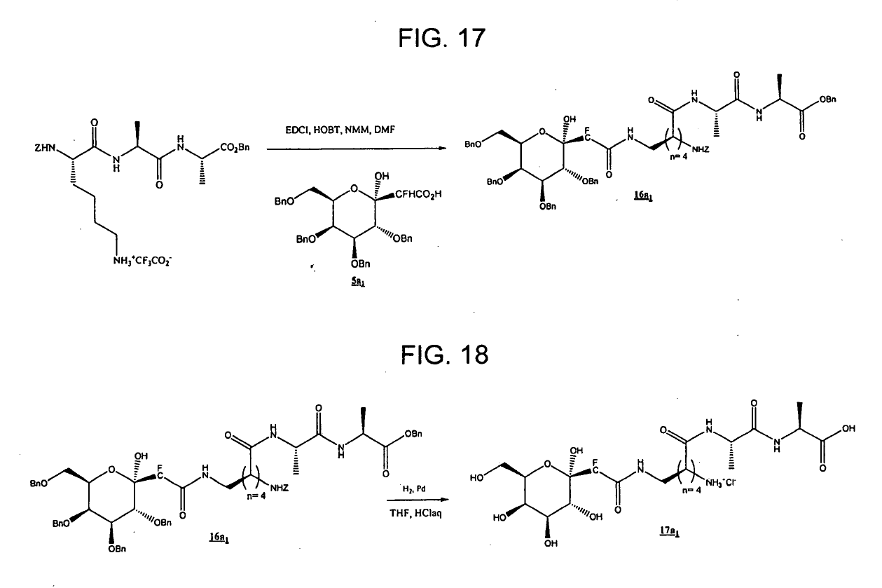

FIG. 17 is a reaction equation for obtaining compound 16a₁;

FIG. 18 is a reaction equation for obtaining compound 17a₁;

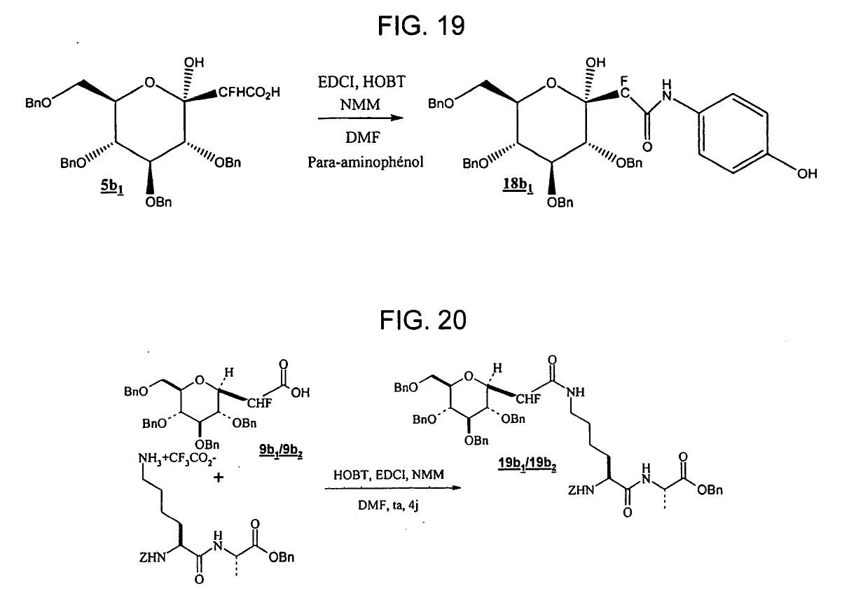

FIG. 19 is a reaction equation for obtaining compound 18b₁;

FIG. 20 is a reaction equation for obtaining compound 19b₁/19b₂;

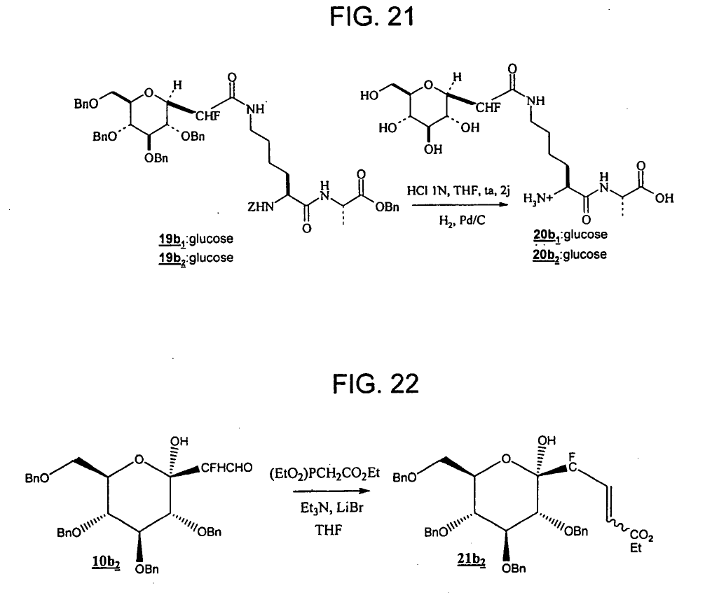

FIG. 21 is a reaction equation for obtaining compound 20b₁/20b₂;

FIG. 22 is a reaction equation for obtaining compound 21b₂;

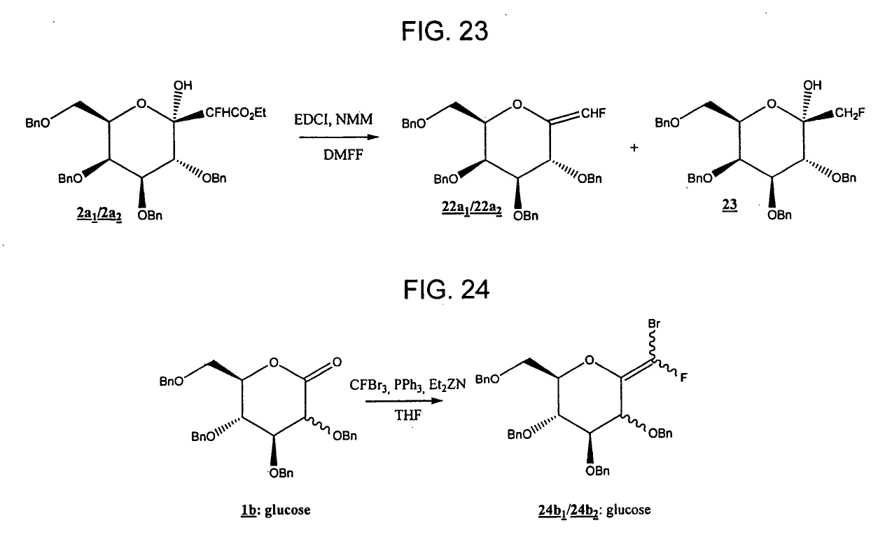

FIG. 23 is a reaction equation for obtaining compound 22a₁/22a₂; 23;

FIG. 24 is a reaction equation for obtaining compound 24b₁/24b₂;

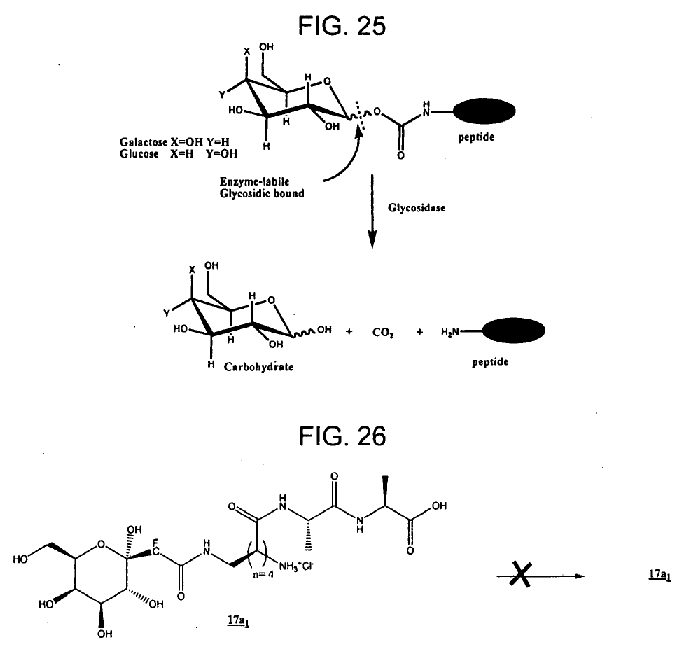

FIG. 25 is an example of enzymatic break-down of O-glycopeptides by glycosidases;

FIG. 26 is an example of resistance of CHF-glycopeptides to glycosidases;

The following abbreviations are used:

eq: equivalent g: gram Hz: Hertz
mg: milligram MHz: megahertz min: minute
ml: milliliter mmol: millimole μmol: micromole
nmol: nanomole de: diastereomeric excess

The characteristics of the devices used to perform the analyses of all the compounds described in the present application are indicated below:

¹H, ¹³C and ¹⁹F NMR spectra were recorded on BRUKER DPX 300 and DPX 600 spectrometers. In ¹H and ¹³C NMR, tetramethylsilane was used as an internal standard. In ¹⁹F NMR, the external standard was fluorotrichloromethane (CFCl₃). Chemical shifts are expressed in parts per million (ppm) and coupling constants (J) in Hertz (Hz).

The following abbreviations were used:

s for singlet, bs for broad singlet, d for doublet, t for triplet, qdt for quadruplet, m for multiplet or mass, dd for doublet of doublet, etc.

Mass spectra were obtained on a Micromass TOF-SPEC spectrophotometer, E 20 kV, α-cyano for matrix-assisted laser desorption/ionization (MALDI) and a JEOL AX500, 3 kV, JEOL FAB gun, Xe, 4 kV, limiting current 10 μA, Gly-NBA 50:50 for FAB ionization.

Separations by column chromatography are performed under light pressure while following chromatography techniques using Kieselgel 60 silica (230-400 mesh, Merck).

Monitoring is by thin layer chromatography (TLC) with Kieselgel 60E-254-0.25 mm plates. Herein, retardation factor (Rf) is defined as the ratio of the migration distance of a compound on a given support to the migration distance of an eluent.

Synthesis of Compounds 2a₁/2a₂ (FIG. 1)

In a flask under inert atmosphere containing previously activated and stripped zinc (2.55 g; 38.96 mmol; 7 eq), THF (40 ml) is added. The mixture is refluxed and then a mixture comprised of lactone 1a (3 g; 5.57 mmol; 1 eq) and ethyl bromofluoroacetate (1.97 ml; 16.7 mmol; 3 eq) in THF (40 ml) is added dropwise. The reaction is refluxed for 3 hours. After the reaction mixture returns to room temperature, a 1 N HCl solution (60 ml) is added. The mixture is filtered on a Buchner funnel to eliminate excess zinc. Dichloromethane (40 ml) is added to the solution. The two phases are separated and the aqueous phase is extracted with dichloromethane two more times. The organic phases are recombined, dried on magnesium sulfate, filtered and then concentrated.

The mixture is then purified on a silica column with as eluent a cyclohexane/ethyl acetate mixture in proportions of 8 to 2 to obtain a colorless oil for the minor diastereoisomer 2a₁ and a light yellow oil for the major diastereoisomer 2a₂ with an overall yield of 70%.

de=38 (69-31) determined by ¹⁹F NMR on the crude reaction product
Characterization of Compounds 2a₁/2a₂

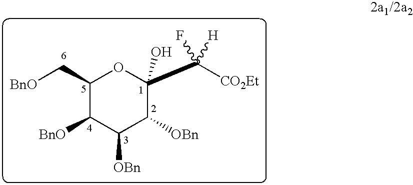

C₃₈H₄₁FO₈ M=644.73 g/mol

### 2a₁—Minor Diastereoisomer

Rf=0.53 (cyclohexane/ethyl acetate 7/3)

¹⁹F NMR (CDCl₃, 282.5 MHz)

−200.5 (dd, J_{F-H}=47 and 2 Hz)

¹H NMR (CDCl₃, 300 MHz)

1.2 (t, 7.2, 3H, CH₃); 3.4 (dd, 5.8-9.21H, H6); 3.6 (dd, 7.7-9.3, 1H, H6); 3.9 (s, 1H, H4; 4 (dd, 2.6-10, 1H, H3); 4.1 (dd, 6.5, 1H, H5); 4.2 (m, 2H, CH₂); 4.3 (d, 11.4, 1H, H2); 4.3-4.9 (m, 8H, 40CH₂Ph); 5 (d, 47 Hz, 1H, CHF); 7.2 (m, 20H, H ar.)

¹³C NMR (CDCl₃, 75.5 MHz)

14.4 (CH₃); 62.9 (CH₂); 69.1 (C6); 71.5 (C5); 73.1 (OCH₂Ph); 73.8 (OCH₂Ph); 74.8 (C4); 74.9 (OCH₂Ph); 75.1 (C2); 76.1 (OCH₂Ph); 80.6 (C3); 86.9 (d, 203 Hz, CHF); 98.5 (d, 21 Hz, Cl); 127.9-128.8 (C ar.); 138.4; 138.5; 138.8; 139.2 (C quat. ar.); 169.5 (d, 22 Hz, CO₂Et).

### 2a₂—Major Diastereoisomer

Rf=0.61 (cyclohexane/ethyl acetate 7/3)

¹⁹F NMR (CDCl₃, 282.5 MHz)

−205.2 (d, J_{F-H}=47 Hz)

¹H NMR (CDCl₃, 300 MHz)

0.9 (t, 7.2, 3H, CH₃); 3.4 (dd, 5.5-9.11H, H6); 3.5 (dd, 9, 1H, H6); 3.6 (s, 1H, OH); 3.9 (qdt, 7.2, 2H, CH₂); 4 (m, 2H, H4, H3); 4.1 (dd, 6.7, 1H, H5); 4.3 (d, 9, 1H, H2); 4.4-5 (m, 8H, 4OCH₂Ph); 4.8 (d, 47 Hz, 1H, CHF); 7.2 (m, 20H, H ar.)

¹³C NMR (CDCl₃, 75.5 MHz)

14.1 (CH₃); 62.3 (CH₂); 68.6 (C6); 71.3 (C5); 72.7 (OCH₂Ph); 73.9 (OCH₂Ph); 74.3 (C4); 75 (OCH₂Ph); 75.2 (C2); 75.4 (OCH₂Ph); 81.1 (C3); 88.7 (d, 193 Hz, CHF); 97.8 (d, 20 Hz, Cl); 127.9-128.9 (C ar.); 138.3; 138.5; 138.6; 139.2 (C quat. ar.); 166.6 (d, 25 Hz, CO₂Et).

Compounds 2a₁/2a₂ can also be obtained according to another synthesis pathway that leads in this case to major diastereoisomer 2a₁ and minor diastereoisomer 2a₂.

Synthesis of Compounds 2b₁/2b₂ (FIG. 1)

In a flask under inert atmosphere containing previously activated and stripped zinc (2.55 g; 38.96 mmol; 7 eq), THF (40 ml) is added. The mixture is refluxed and then a mixture comprised of lactone 1b (3 g; 5.57 mmol; 1 eq) and ethyl bromofluoroacetate (1.97 ml; 16.7 mmol; 3 eq) in THF (40 ml) is added dropwise. The reaction is refluxed for 3 hours. After the reaction mixture returns to room temperature, a 1 N HCl solution (60 ml) is added. The mixture is filtered on a Buchner funnel to eliminate excess zinc. Dichloromethane (40 ml) is added to the solution. The two phases are separated and the aqueous phase is extracted with dichloromethane two more times. The organic phases are recombined, dried on magnesium sulfate, filtered and then concentrated.

The mixture is then purified on a silica column with as eluent a cyclohexane/ethyl acetate mixture in proportions of 9.3 to 0.7 to obtain white crystals for the minor diastereoisomer 2b₁ and a light yellow oil for the major diastereoisomer 2b₂ with an overall yield of 61%.

de=46 (73-27) by ¹⁹F NMR

de=54 (77-23) by HPLC (Kromasil C18 column, UV 254 nm, 90/10 CH₃CN/H₂O, 1 ml/min)

Characterization of 2b₁/2b₂

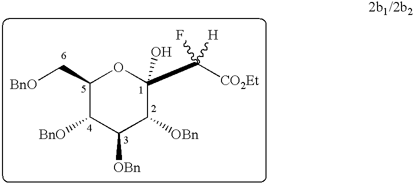

C₃₈H₄₁FO₈ M=644.73 g/mol

### 2b₁—Minor Diastereoisomer

Rf=0.23 (cyclohexane/ethyl acetate 8/2).

¹⁹F NMR (CDCl₃, 282.5 MHz)

−200.2 (d, J_{F-H}=47 Hz)

¹H NMR (CDCl₃, 300 MHz)

1.2 (t, 7.2, 3H, CH₃); 3.5 (dd, 1.5-12.3, 1H, H6); 3.6 (dd, 9.8, 1H, H4); 3.65 (dd, 4.6-11.4, 1H, H6); 3.7 (dd, 1.5-9.7, 1H, H2); 4.0 (dd, 2.7-10, 1H, H5); 4.1 (dd, 9.7, 1H, H3); 4.2 (m, 2H, CH₂); 4.4-4.9 (m, 8H, 4OCH₂Ph); 4.9 (d, 44, 1H, CHF); 7.2 (m, 20H, H ar.)

¹³C NMR (CDCl₃, 75.5 MHz)

14.5 (CH₃); 63.0 (CH₂); 69.0 (C6); 72.9 (C5); 73.7 (OCH₂Ph); 75.5 (OCH₂Ph); 76 (OCH₂Ph); 76.1 (OCH₂Ph); 78.5 (C2); 78.7 (C4); 83 (C3); 86.9 (d, 203 Hz, CHF); 98 (d, 21 Hz, Cl); 127.9-128.9 (C ar); 138.2; 138.5; 138.8; 138.9 (C quat. ar.); 163.3 (d, 23 Hz, CO₂Et).

### 2b₂—Major Diastereoisomer

Rf=0.17 (cyclohexane/ethyl acetate 8/2).

¹⁹F NMR (CDCl₃, 282.5 MHz)

−205.5 (dd, J_{F-H}=47 and 11 Hz)

¹H NMR (CDCl₃, 300 MHz) 1.1 (t, 7.2, 3H, CH₃); 3.6 (m, 1H, H6); 3.7 (m, 2H, H4, H6); 3.8 (d, 9.4, 1H, H2); 3.9 (m, 3H, H5.2 CH₂); 4.1 (dd, 9.4, 1H, H3); 4.4-5 (m, 8H, 4OCH₂Ph); 4.8 (d, 47 Hz, 1H, CHF); 7.2 (m, 20H, H ar.)

¹³C NMR (CDCl₃, 75.5 MHz)

14.4 (CH₃); 62.5 (CH₂); 68.7 (C6; 72.9 (C5); 73.7 (OCH₂Ph); 75.3 (OCH₂Ph); 75.5 (OCH₂Ph); 76.1 (OCH₂Ph); 78.4 (C4); 78.7 (C2); 83.6 (C3); 86.8 (d, 195 Hz, CHF); 97.5 (d, 20 Hz, Cl); 127.9-129.9 (C ar.); 138.3; 138.5; 138.7; 138.8 (C quat. ar.); 166.8 (d, 24 Hz, CO₂Et).

Compounds 2b₁/2b₂ can also be obtained according to another synthesis pathway which in this case leads to major diastereoisomer 2b₁ and minor diastereoisomer 2b₂.

Synthesis of Compounds 2c₁/2c₂ (FIG. 1)

In a flask under inert atmosphere containing previously activated and stripped zinc (2.55 g; 38.96 mmol; 7 eq), THF (40 ml) is added. The mixture is refluxed and then a mixture comprised of lactone 1c (3 g; 5.57 mmol; 1 eq) and ethyl bromofluoroacetate (1.97 ml; 16.7 mmol; 3 eq) in THF (40 ml) is added dropwise. The reaction is refluxed for 3 hours. After the reaction mixture returns to room temperature, a 1 N HCl solution (60 ml) is added. The mixture is filtered on a Buchner funnel to eliminate excess zinc. Dichloromethane (40 ml) is added to the solution. The two phases are separated and the aqueous phase is extracted with dichloromethane two more times. The organic phases are recombined, dried on magnesium sulfate, filtered and then concentrated.

The mixture is then purified on a silica column with as eluent a cyclohexane/ethyl acetate mixture in proportions of 8.5 to 1.5 to obtain white crystals for the major diastereoisomer 2c₁ and a light yellow oil for the minor diastereoisomer 2c₂ with an overall yield of 67% and a de=56 (78-22) by ¹⁹F NMR.

Characterization of 2c₁/2c₂

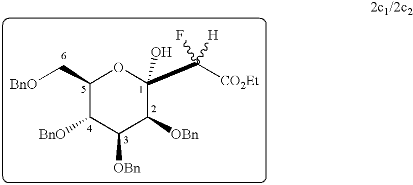

C₃₈H₄₁FO₈ M=644.73 g/mol

### 2c₁—Major Diastereoisomer

Rf=0.53 (cyclohexane/ethyl acetate 7/3).

¹⁹F NMR (CDCl₃, 282.5 MHz)

−200.0 (d, J_{F-H}=47 Hz)

¹H NMR (CDCl₃, 300 MHz)

1.1 (t, 7.2, 3H, CH₃); 3.5 (dd, 1.1-11.5 Hz, 1H, H6) 3.7 (dd, 4.4-11.5 Hz, 1H, H6); 3.9 (bs, 1H, H2); 4.0 (m, 2H, H5, H4); 4.1 (dd, 2.4-9.5 Hz, 1H, H3); 4.1 (dqdt, 2.3-7.2 Hz, 2H, CH₂); 4.3-4.9 (m, 8H, 40CH₂Ph); 5.0 (d, 48 Hz, 1H, CHF); 7.2 (m, 20H, H ar.).

¹³C NMR (CDCl₃, 75.5 MHz)

14.4 (CH₃); 62.7 (CH₂); 69.6 (C6); 73.1 (OCH₂Ph); 73.9 (OCH₂Ph); 74.0 (C5); 75.1 and 75.2 (C4 and C2; 75.4 (OCH₂Ph); 75.6 (OCH₂Ph); 81.6 (C3); 85.6 (d, 181 Hz, CHF); 97.9 (d, 26 Hz, Cl); 128-128.9 (C ar.); 138.7; 138.8; 138.9; 139.0 (C quat. ar.); 169.3 (d, 23 Hz, CO₂Et).

### 2c₂—Minor Diastereoisomer

Rf=0.44 (cyclohexane/ethyl acetate 8/2).

¹⁹F NMR (CDCl₃, 282.5 MHz)

−210.3 (d, J_{F-H}=48 Hz)

¹H NMR (CDCl₃, 300 MHz)

1.2 (t, 7 Hz, 3H, CH₃); 3.7 (d, 2.9 Hz, 2H, H6); 3.9 (td, 3.2-9.4 Hz, 1H, H5); 4 (t, 9.2 Hz, 1H, H4); 4 (m, 2H, H2, H3); 4.1 (m, 2H, CH₂); 4.4-5 (m, 8H, 4OCH₂Ph); 5.2 (d, 48 Hz, 1H, CHF); 7.2 (m, 20H, H ar.)

¹³C NMR (CDCl₃, 75.5 MHz)

14.5 (CH₃); 62.2 (CH₂); 69.5 (C6); 73.2 (OCH₂Ph); 73.8 (OCH₂Ph); 74.2 (C5); 74.4 (OCH₂Ph); 75.2 (C4); 75.6 (OCH₂Ph); 76 (C2); 82 (C3); 90 (d, 192 Hz, CHF); 97.9 (d, 19 Hz, Cl); 127.7-128.8 (C ar.); 138.7; 138.8; 138.9; 139.3 (C quat. ar.); 167.1 (d, 24 Hz, CO₂Et).

Synthesis of Compounds 2a₁/2a₂ and 3a₁/3a₂ (FIG. 2)

To a solution of triphenylphosphine (0.5 g, 2 mmol, 4 eq) in anhydrous THF (5 ml) placed under inert atmosphere lactone 1a (269 mg, 0.5 mmol, 1 eq) solubilized in 2 ml of THF is added. Et₂Zn (C=1.0 M in hexane, 2 mmol, 4 eq) and ethyl dibromofluoroacetate (0.14 ml, 1 mmol, 2 eq) are then added successively to the mixture. The mixture is stirred at room temperature for three hours, before being hydrolyzed by an NH₄Cl saturated aqueous solution. The salts are then filtered on celite and the filtrate is evaporated under reduced pressure.

The reaction mixture reveals the presence of two products: 90% products 2a₁/2a₂ in the form of two diastereoisomers: major diastereoisomer 2a₁ and minor diastereoisomer 2a₂ (de: 94/6 determined by ¹⁹F NMR) and 10% products 3a₁/3a₂ in the form of two isomers (de=60 (80-20) by ¹⁹F NMR).

Products 2a₁/2a₂ are purified on a silica gel with as eluent a mixture of cyclohexane/ethyl acetate in proportions of 8 to 2 with an overall yield of 56%. Products 2a₁/2a₂ have been characterized above.

Products 3a₁/3a₂ are purified on a silica gel with as eluent a mixture of cyclohexane/ethyl acetate in proportions of 9 to 1 to give a mixture of the two diastereoisomers with a yield of 20%.

Characterization of Compounds 3a₁/3a₂

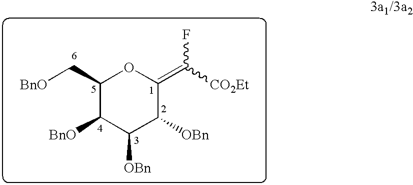

C₃₈H₃₉FO₇ M=626.73 g/mol

Rf=0.6 (cyclohexane/ethyl acetate 8/2),

¹⁹F NMR (CDCl₃, 282.5 MHz):

Major Diastereoisomer 3a₁:

−144.2 ppm

Minor Diastereoisomer 3a₂:

−144.6 ppm

¹H NMR (CDCl₃, 300 MHz):

1.2 (2t, 0.8H, J=7.2 Hz, CH₃); 3.8-3.6 (m, 3H, H6 and H4); 3.8 (m, 1H, H3); 4.1 (dq, 2H, J=7.3 Hz, CH₂); 4.3 (m, 1H, H5); 4.6-4.4 (m, 8H, 4 OCH₂Ph); 7.3 (m, 20H, Har.).

¹³C NMR (CDCl₃, 75.5 MHz):

14.4 (CH3a₂); 14.6 (CH3a₁; 61.8 (CH₂a₁); 63.1 (CH₂a₂); 69.2 (C6); 70.8 (C2); 71.2 (CH₂-Ph); 72.0 (CH₂-pH); 72.5 (CH₂-pH); 73.8 (CH₂-pH); 70.8 (C4); 72.8 (C5); 78.4 (C3a₁); 79.4 (C3a₂); 128.8-127.8 (20 Car.); 138.7-138.2 (C quat. ar.); 147.7 (d, J=7.92 Hz, Cl); 162.1 (d, J=26.87 Hz, CO₂Et).

Synthesis of Compounds 2b₁/2b₂ and 3b₁ (FIG. 2):

To a solution of triphenylphosphine (0.5 g, 2 mmol, 4 eq) in anhydrous THF (5 ml) placed under inert atmosphere lactone 1b (269 mg, 0.5 mmol, 1 eq) solubilized in 2 ml of THF is added. Et₂Zn (C=1.0 M in hexane, 2 mmol, 4 eq) and ethyl dibromofluoroacetate (0.14 ml, 1 mmol, 2 eq) are then added successively to the mixture. The mixture is stirred at room temperature for three hours, before being hydrolyzed by an NH₄Cl saturated aqueous solution. The salts are then filtered on celite and the filtrate is evaporated under reduced pressure.

The crude mixture reveals the presence of two products: 90% products 2b₁/2b₂ in the form of two diastereoisomers (de: 91/9) and 10% product 3b₁ in the form of a single isomer.

Products 2b₁/2b₂ are purified on a silica gel with as eluent a mixture of cyclohexane/ethyl acetate in proportions of 8.5 to 1.5 to obtain a compound in the form of a mixture two diastereoisomers 2b₁/2b₂ with a yield of 58%. Under these reaction conditions compound 2b₁ is the major diastereoisomer and 2b₂ the minor diastereoisomer. Products 2b₁/2b₂ have been characterized above.

Product 3b₁ is purified on a silica gel with as eluent a cyclohexane/ethyl acetate mixture in proportions of 9.8 to 0.2 to obtain a compound in the form of a single isomer with a yield of 20%.

Characterization of Compound 3b₁

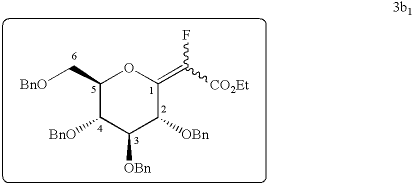

C₃₈H₃₉FO₇ M=626.73 g/mol

Rf=0.4 (cyclohexane/ethyl acetate 8/2).

¹⁹F NMR (CDCl₃, 282.5 Mz)

−149.8 ppm

¹H NMR (CDCl₃, 300 Mz):

1.2 (t, 0.8H, J=7.2 Hz, CH₃; 3.8-3.6 (m, 3H, H6 and H4); 3.8 (m, 1H, H3); 4.1 (dq, 2H, J=7.0-1.9 Hz, CH₂); 4.3 (m, 1H, H5); 4.6-4.4 (m, 8H, 4 OCH₂Ph); 5.6 (s, H2) 7.3 (m, 20H, Har.).

¹³C NMR (CDCl₃, 75.5 Mz):

14.6 (CH₃); 61.4 (CH₂); 68.6 (C6); 70.7 (C2); 71.0 (OCH₂Ph); 71.5 (OCH₂Ph); 73.0 (OCH₂Ph); 73.7 (OCH₂Ph); 76.1 (C5); 78.2 (C4); 81.4 (C3); 128.8-128.1 (C ar.); 138.6; 138.2; 138.1; 137.6; 137.3 (Car.q.); 137.3 (d, J=252 Hz, CF); 147.5 (d, J=8 Hz, CD; 162.5 (d, J=27 Hz, CO₂Et).

Synthesis of Compounds 2c₁/2c₂ and 3c₁/3c₂ (FIG. 2)

To a solution of triphenylphosphine (0.5 g, 2 mmol, 4 eq) in anhydrous THF (5 ml) placed under inert atmosphere lactone 1c (269 mg, 0.5 mmol, 1 eq) solubilized in 2 ml of THF is added. Et₂Zn (C=1.0 M in hexane, 2 mmol, 4 eq) and ethyl dibromofluoroacetate (0.14 ml, 1 mmol, 2 eq) are then added successively to the mixture. The mixture is stirred at room temperature for three hours, before being hydrolyzed by an NH₄Cl saturated aqueous solution. The salts are then filtered on celite and the filtrate is evaporated under reduced pressure.

The crude mixture reveals the presence of two products: 88% products 2c₁/2c₂ in the form of two diastereoisomers (de: 75/25) and 12% products 3c₁/3c₂ in the form of two isomers (78/22).

Products 2c₁/2c₂ are purified on a silica gel with as eluent a mixture of cyclohexane/ethyl acetate in proportions of 8.5 to 1.5 to obtain compound 2c₁/2c₂ in the form of two diastereoisomers with a yield of 66%.

In this case the method leads to the same major diastereoisomer 2c₁ and minor diastereoisomer 2c₂ as with the preceding method. Products 2c₁/2c₂ have been characterized above.

Products 3c₁/3c₂ are purified on a silica gel with as eluent a mixture of cyclohexane/ethyl acetate in proportions of 9 to 1 to obtain compound in the form of a mixture of two isomers (de=78/22 by ¹⁹F NMR) with a yield of 12%.

Characterization of Compounds 3c₁/3c₂

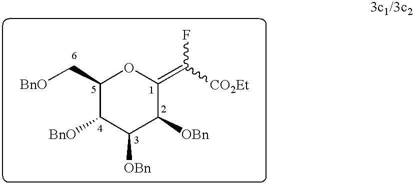

C₃₈H₃₉FO₇ M=626.73 g/mol

Rf=0.6 (cyclohexane/ethyl acetate 8/2)

¹⁹F NMR (CDCl₃, 282.5 MHz):

Minor Diastereoisomer 3c₁:

−143.9 ppm

Major Diastereoisomer 3c₂:

−149.5 ppm

¹H NMR (CDCl₃, 300 MHz):

1.2 (t, 1H, J=7.1 Hz, CH₃); 3.8-3.6 (m, 3H, H6 and H4); 3.9 (t, J=4.2 Hz, H3); 4.1-4.3 (m, 2H, CH₂); 4.6-4.4 (m, 8H, 4 OCH₂Ph); 4.9 (td, 1H, J=6.64, 4.47, 4.47 Hz, H5); 5.71 (dd, 1H, J=3.31, 2.60 Hz, H2); 7.3 (m, 20H, H ar.).

¹³C NMR (CDCl₃, 75.5 MHz):

14.6 (CH₃); 61.7 (CH₂); 68.2 (C2); 68.8 (C6); 71.0 (CH₂-pH); 71.6 (CH₂-pH); 72.9 (CH₂-pH); 73.9 (CH₂-pH); 78.0 (C4); 78.2 (C5); 79.9 (d, J=3.6 Hz, C3); 128.8-128.0 (20 Car.); 138.4; 138.3; 138.2; 137.7 (4 Car.quat.); 136.47 (d, J=254.24 Hz, CF); 147.89 (d, J=7.92 Hz, Cl); 162.43 (d, J=26.87 Hz, CO₂Et).

Synthesis of Compounds 4a₁/4a₂ (FIG. 3):

In a flask under inert atmosphere containing lactone 1a (269 mg, 0.5 mmol, 1 eq) in solution in THF (5 ml), diethylzinc (Et₂Zn) (C=1.0 M in hexane, 1 mmol, 2 eq) and ethyl dibromofluoroacetate (0.14 ml, 1 mmol, 2 eq) are added successively to the mixture. The solution is then stirred at room temperature for three hours before being hydrolyzed with ethanol and evaporated under reduced pressure.

The mixture is then purified on a silica gel with as eluent a mixture of cyclohexane/ethyl acetate in proportions of 9.5 to 0.5 to obtain compounds 4a₁/4a₂ in the form of a mixture of two diastereoisomers (de=50 (75-25) by ¹⁹F NMR) with a yield of 62%.

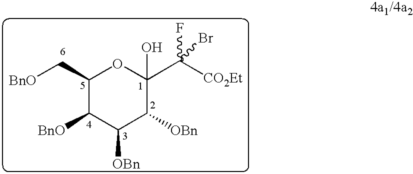

C₃₈H₄₀FBrO₈ M=723.74 g/mol

Rf=0.4 (cyclohexane/ethyl acetate 8/2)

¹⁹F NMR (CDCl₃, 282.5 MHz):

Major Diastereoisomer 4a₁:

−126.5 ppm

Minor Diastereoisomer 4a₂:

−126.7 ppm

¹H NMR (CDCl₃, 300 MHz):

1.1 (t, 0.8H, J=7.2 Hz, CH₃a₂); 2.2 (t, 1.2H, J=7.2 Hz, CH₃a₁); 3.6-3.5 (dd, 1H, J=7-12 Hz, H6); 3.9-3.7 (dd, 1H, J=7.3 Hz, H6); 4.0 (s, 1H, H4); 4.0 (m, 1H, H3); 4.0 (m, 1H, H5); 4.1 (q, 2H, J=7, 0.3 Hz, CH₂); 4.2 (m, 1H, H2); 4, −4.8 (m, 8H, 4 OCH₂Ph); 7.3 (m, 20H, Har.).

¹³C NMR (CDCl₃, 75.5 MHz)

Major Diastereoisomer 4a₁:

14.2 (CH₃); 64.0 (CH₂); 68.9 (C6); 72.4 (C5); 73.5 (OCH₂Ph); 73.8 (OCH₂Ph); 74.4 (C4); 74.9 (OCH₂Ph); 75.5 (C2); 75.7 (OCH₂Ph); 81.4 (C3); 98.0 (d, J=274 Hz, CF); 98.5 (d, J=25 Hz, Cl); 127-129 (C ar.); 139-138 (Car.quat.); 166.4 (d, J=26 Hz, CO₂Et).

Minor Diastereoisomer 4a₂:

13.9 (CH₃); 63.8 (CH₂); 68.7 (C6); 72.2 (C5); 72.8 (OCH₂Ph); 74.0 (OCH₂Ph); 74.8 (OCH₂Ph); 75.0 (C4); 75.1 (OCH₂Ph); 75.5 (C2); 81.9 (C3); 99.0 (d, J=295 Hz, CF); 98.0 (d, J=23 Hz, Cl); 127-129 (C ar.); 139-138 (Car.quat.); 165.6 (d, J=26 Hz, CO₂Et).

Synthesis of Compounds 4b₁/4b₂ (FIG. 3):

In a flask under inert atmosphere containing lactone 1b (269 mg, 0.5 mmol, 1 eq) in solution in THF (5 ml), Et₂Zn (C=1.0 M in hexane, 1 mmol, 2 eq) and ethyl dibromofluoroacetate (0.14 ml, 1 mmol, 2 eq) are added successively to the mixture. The solution is then stirred at room temperature for three hours before being hydrolyzed with ethanol and evaporated under reduced pressure.

The mixture is then purified on a silica gel with as eluent a mixture of cyclohexane/ethyl acetate in proportions of 9.5 to 0.5 to obtain compounds 4b₁/4b₂ in the form of a mixture of two diastereoisomers (de=(92-8) by ¹⁹F NMR) with a yield of 41%.

Characterization of Compounds 4b₁/4b₂

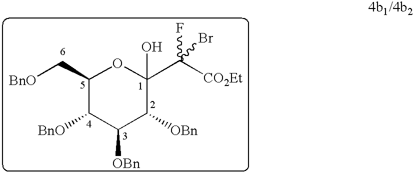

C₃₈H₄₀FBrO₈ M=723.74 g/mol

Rf=0.37 (cyclohexane/ethyl acetate 8/2)

¹⁹F NMR (CDCl₃, 282.5 MHz):

Minor Diastereoisomer 4b₁:

−127.2 ppm

Major Diastereoisomer 4b₂:

−127.8 ppm

¹H NMR (CDCl₃, 300 MHz):

1.1 (t, 0.8H, J=7.2 Hz; 2.2 (t, 1.2H, J=7.2 Hz, CH₃b₂) 3.6-3.5 (dd, 1H, J=7-12 Hz, H6); 3.9-3.7 (dd, 1H, J=7.3 Hz, H6); 4.0 (s, 1H, H4); 4.0 (m, 1H, H3); 4.0 (m, 1H, H5); 4.1 (q, 2H, J=7.3 Hz, CH₂); 4.2 (m, 1H, H2); 4.3-4.8 (m, 8H, 4 OCH₂Ph); 7.3 (m, 20H, Har.).

¹³C NMR (CDCl₃, 75.5 MHz):

Minor Diastereoisomer 4b₁:

13.9 (CH₃); 63.8 (CH₂); 68.7 (C6); 72.2 (C5); 72.8 (OCH₂Ph); 74.0 (OCH₂Ph); 74.8 (OCH₂Ph); 75.0 (C4); 75.1 (OCH₂Ph); 75.5 (C2); 81.9 (C3); 99.0 (d, J=295 Hz, CF); 98.0 (d, J=23 Hz, Cl); 127-129 (C ar.); 139-138 (Car.quat.); 165.6 (d, J=26 Hz, CO₂Et).

Major Diastereoisomer 4b₂:

14.2 (CH₃); 64.0 (CH₂); 68.9 (C6); 72.4 (C5); 73.5 (OCH₂Ph); 73.8 (OCH₂Ph); 74.4 (C4); 74.9 (OCH₂Ph); 75.5 (C2); 75.7 (OCH₂Ph); 81.4 (C3); 98.0 (d, J=274 Hz, CF; 98.5 (d, J=25 Hz, Cl); 127-129 (C ar.); 139-138 (Car.quat.); 166.4 (d, J=26 Hz, CO₂Et).

Synthesis of Compounds 4c₁/4c₂ (FIG. 3)

In a flask under inert atmosphere containing lactone 1c (269 mg, 0.5 mmol, 1 eq) in solution in THF (5 ml), Et₂Zn (C=1.0 M in hexane, 1 mmol, 2 eq) and ethyl dibromofluoroacetate (0.14 ml, 1 mmol, 2 eq) are added successively to the mixture. The solution is then stirred at room temperature for three hours before being hydrolyzed with ethanol and evaporated under reduced pressure.

The mixture is then purified on a silica gel with as eluent a mixture of cyclohexane/ethyl acetate in proportions of 9.5 to 0.5 to obtain compound in the form of a mixture of two diastereoisomers 4c₁/4c₂ (de=(42-58) by ¹⁹F NMR) with a yield of 43%.

Characterization of Compounds 4c₁/4c₂

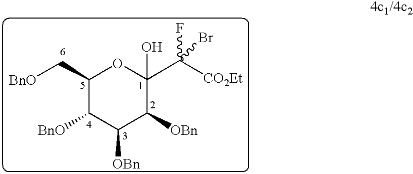

C₃₈H₄₀FBrO₈ M=723.74 g/mol

Rf=0.34 (cyclohexane/ethyl acetate 8/2)

¹⁹F NMR (CDCl₃, 282.5 MHz):

Minor Diastereoisomer 4c₁:

−120.3 ppm

Major Diastereoisomer 4c₂:

−124.9 ppm

¹H NMR (CDCl₃, 300 MHz):

0.9 (t, 1H, J=7.1 Hz; CH₃c₂) 1.1 (t, 1.2H, J=7.1 Hz, CH₃Cl); 3.6-3.5 (dd, 1H, J=7-12 Hz, H6); 3.9-3.7 (dd, 1H, J=7.3 Hz, H6); 4.0 (s, 1H, H4); 4.0 (m, 1H, H4); 4.0 (m, 1H, H5); 4.1 (q, 2H, J=7.3 Hz, CH₂); 4.2 (m, 1H, H2) 4.3-4.9 (m, 8H, 4 OCH₂Ph); 7.3-7.1 (m, 20H, H ar.).

¹³C NMR (CDCl₃, 75.5 MHz):

Minor Diastereoisomer 4c₁:

14.1 (CH₃); 64.0 (CH₂); 69.2 (CH₂); 73.4 (OCH₂Ph); 74.0 (OCH₂Ph); 74.6 (C5); 74.8 (OCH₂Ph); 75.1 (C4); 75.6 (OCH₂Ph); 76.7 (C2); 82.2 (C3); 98.2 (d, J=20 Hz, C1); 103.2 (d, J=282 Hz, CF); 128.9-127.8 (C ar.); 139.1-138.6 (Car.quat.); 165.0 (d, J=25 Hz, CO₂Et).

Major Diastereoisomer 4c₂:

14.0 (CH₃); 63.6 (CH₂); 69.5 (C6); 73.3 (OCH₂Ph); 73.8 (OCH₂Ph); 74.8 (OCH₂Ph); 74.9 (C5); 75.0 (C2); 75.6 (OCH₂Ph); 82.0 (C3); 98.9 (d, J=25 Hz; 100.6 (d, J=272 Hz, CF); 127-129 (C ar.); 139-138 (Car.quat.); 165.7 (d, J=27 Hz, CO₂Et)

Synthesis of Compounds 5a₁/5a₂ (FIG. 4)

In a flask containing ester 2a₁/2a₂ (500 mg; 0.776 mmol; 1 eq) in solution in THF (5 ml) is added a solution comprised of LiOH (37 mg; 1.55 mmol; 2 eq) solubilized in a minimum of water. The reaction is stirred overnight. A 1 M HCl solution is added and the mixture is extracted three times with ethyl acetate. The organic phases are recombined, dried on magnesium sulfate, filtered and then evaporated to obtain the expected product in the form of a yellow oil with a yield of 96%.

Characterization of Compounds 5a₁/5a₂

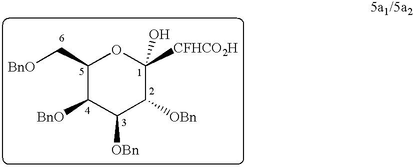

C₃₆H₃₇FO₈ M=616.67 g/mol

¹⁹F NMR (CDCl₃, 282.5 MHz):

−197.9 (d, J_{F-H}=49 Hz, 1F): 5a₁

−202.7 (d, J_{F-H}=47 Hz, 1F): 5a₂

¹H NMR (CDCl₃, 300 MHz) 3.4 (dd, 2.2-6.2 2H, H6); 3.8 (s, 1H, H4); 3.9 (dd, 2.4 and 10 Hz, 1H, H3); 4.1 (tapp., 6.1 Hz, 1H, H5); 4.2 (dd, 2.8 and Hz, 1H, H2); 4.3 (d, 11.9 Hz, 1H, OCH₂Ph); 4.4 (d, 12 Hz, 1H, OCH₂Ph); 4.5 (d, 11.7 Hz, 1H, OCH₂Ph); 4.6 (d, 11.1 Hz, 1H, OCH₂Ph); 4.7 (s, 2H, OCH₂Ph); 4.8 (d, 12 Hz, 1H, OCH₂Ph); 4.9 (d, 11.2 Hz, 1H, OCH₂Ph); 4.9 (d, 47 Hz, 1H, CHF); 7.2 (m, 20H, H ar.).

¹³C NMR (CDCl₃, 75.5 MHz)

69.2 (C6); 71.5 (C5); 73.2 (OCH₂Ph); 73.7 (OCH₂Ph); 74.8 (OCH₂Ph); 74.9 (C4); 75.2 (C2); 76.2 (OCH₂Ph); 80.6 (C3); 86.0 (d, 200 Hz, CHF); 98.4 (d, 21 Hz, Cl); 128.0-128.9 (C ar.); 137.9; 138.3; 138.7; 139.0 (C quat. ar.); 171.0 (d, 24 Hz, CO₂H).

Synthesis of Compound 5b₁ (FIG. 4)

In a flask under inert atmosphere containing ester 2b₁ (275 mg; 0.43 mmol; 1 eq) in solution in ethanol (7 ml), an aqueous lithium hydroxide solution (2 M; 2 eq) is added and the mixture is stirred overnight at room temperature. The mixture is concentrated and dissolved in DCM (5 ml), and then acidified with a 1 M HCl solution (20 ml). The mixture is extracted with DCM (3×20 ml) and the organic phases are combined, washed with a saturated NaCl solution and concentrated directly. Acid 5b₁ is isolated as a yellow solid, which can be used directly for the next step without additional purifications, with a crude yield of 92%.

Characterization of Compound 5b₁

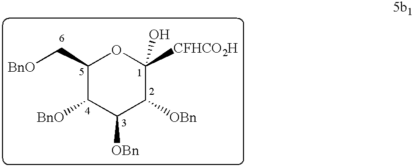

¹⁹F NMR (CDCl₃, 282 MHz)

−197.9 (d, J_{F-H} 46.1)

¹H NMR (CDCl₃, 300 MHz)

3.42-3.71 (m, 4H), 3.94-4.09 (m, 2H), 4.29-4.91 (m, 9H), 7.03-7.28 (m, 20H, H_{Ar})

Synthesis of Compound 6a₂ (FIG. 5)

In a flask under inert atmosphere containing monofluoroester 2a₂ (230 mg; 0.36 mmol; 1 eq) in solution in anhydrous dichloromethane (3 ml) at −30° C., SOBr₂ (41 μl; 0.535 mmol; 1.5 eq) is added dropwise. After 30 minutes, pyridine (42 μl; 0.535 mmol; 1.5 eq) is added and the mixture is stirred for an additional 30 minutes at −30° C. A 2 M HCl solution is added and the phase is extracted three times with dichloromethane. The organic phases are recombined, dried on MgSO₄, filtered and then concentrated under reduced pressure. The crude product is obtained in the form of a light yellow oil with a yield of 80%.

Characterization of Compound 6a₂

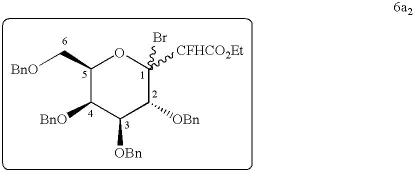

C₃₈H₄₀BrFO₇ M=706.62 g/mol

¹⁹F NMR (CDCl₃, 282 MHz)

−188.0 (d, 45 Hz)

¹H NMR (CDCl₃, 300 MHz)

0.97 (t, 7.1 Hz, 3H, CH₃); 3.51 (dd, 5.4 and 9.1 Hz, 1H, 1H6); 3.60 (tapp, 8.6 Hz, 1H, 1H6); 3.91-4.1 (m, 5H, H4, H3, CH₂, H5); 4.22 (d, 9.4 Hz, 1H, H2); 4.37 (d, 11.8 Hz, 1H, OCH₂Ph); 4.43 (d, 11.8 Hz, 1H, OCH₂Ph); 4.45 (d, ¹Hz, 1H, OCH₂Ph); 4.63 (d, 11.5 Hz, 1H, OCH₂Ph); 4.68 (d, 11.8 Hz, 1H, OCH₂Ph); 4.78 (d, 11.6 Hz, 1H, OCH₂Ph); 4.86 (d, 11 Hz, 1H, OCH₂Ph); 5.01 (d, 11.5 Hz, 1H, OCH₂Ph); 5.07 (d, 46 Hz, 1H, CHF); 7.2 (m, 20H, H ar.)

¹³C NMR (CDCl₃, 75.5 MHz)

14.2 (CH₃); 62.7 (CH₂); 67.4 (C6); 73.0 (OCH₂Ph); 73.6 (C4); 74.0 (OCH₂Ph); 75.0 (OCH₂Ph); 75.3 (OCH₂Ph); 76.2 (C2); 76.4 (C5); 82.3 (C3); 89.5 (d, 199 Hz, CHF); 106.9 (d, 18 Hz, Cl); 127.5-128.9 (C ar.); 138.0; 138.3; 138.7; 138.9 (C quat. ar.); 164.9 (d, 27 Hz, CO₂Et).

Synthesis of Compound 6b₂ (FIG. 5)

In a flask under inert atmosphere containing monofluoroester 2b₂ (500 mg; 0.775 mmol; 1 eq) in solution in anhydrous dichloromethane (6 ml) at −30° C., SOBr₂ (88 μl; 1.13 mmol; 1.5 eq) is added dropwise. After 30 minutes, pyridine (92 μl; 1.13 mmol; 1.5 eq) is added and the mixture is stirred for an additional 30 minutes at −30° C. A 2 M HCl solution is added and the phase is extracted three times with dichloromethane. The organic phases are recombined, dried on MgSO₄, filtered and then concentrated under reduced pressure.

Crude product 6b₂ is obtained in the form of a brown oil with a yield of 85%.

Characterization of Compound 6b₂

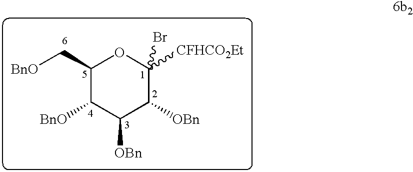

C₃₈H₄₀BrFO₇ M=706.62 g/mol

¹⁹F NMR (CDCl₃, 282 MHz)

−188.09 (d, 45 Hz)

¹H NMR (CDCl₃, 300 MHz)

1.1 (t, 7.1 Hz, 3H, CH₃); 3.6 (dd, 1.7-11.5 Hz, 1H, 1H6) 3.7 (m, 2H, H2, 1H6); 3.8 (dd, 9 Hz, 1H, H4); 3.9 (td, 2.2-10.1 Hz, 1H, H5); 4.1 (qdt, 7, 0.1 Hz, 2H, CH₂); 4.1 (dd, 9.2, 1H, H3); 4.4-5 (m, 8H, 40CH₂Ph); 5.06 (d, 46 Hz, 1H, CHF); 7.2 (m, 20H, H ar.)

¹³C NMR (CDCl₃, 75.5 MHz)

14.4 (CH₃); 62.8 (CH₂); 67.7 (C6); 73.7 (OCH₂Ph); 75 (OCH₂Ph); 75.7 (OCH₂Ph); 76.2 (OCH₂Ph); 76.6 (C2); 78.1 (C5); 79.9 (C4); 84.7 (C3); 89.5 (d, 203 Hz, CHF); 105.7 (d, 18 Hz, Cl); 127.5-128.9 (C ar.); 138.2; 138.4; 138.5 (C quat. ar.); 165.4 (d, 26 Hz, CO₂Et).

The same reaction carried out under the same conditions with compound 2b₁ leads to compound 6b₁.

¹⁹F NMR (CDCl₃, 282 MHz)

−182.2 (d, 47 Hz).

Synthesis of Compound 7a₂ (FIG. 6)

In a flask under inert atmosphere containing monofluoroester 2a2 (95 mg; 0.147 mmol: 1 eq) in solution in anhydrous dichloromethane (2 ml) at −30° C., thionyl chloride (SOCl₂) (16 μl; 0.221 mmol; 1.5 eq) is added dropwise. After 30 minutes, pyridine (17 μl; 0.221 mmol; 1.5 eq) is added and the mixture is stirred for an additional 30 minutes at −30° C. A 2 M HCl solution is added and the phase is extracted three times with dichloromethane. The organic phases are recombined, dried on MgSO₄, filtered and then concentrated under reduced pressure. The crude product is obtained in the form of a light yellow oil with a yield of 83%.

Characterization of Compound 7a2

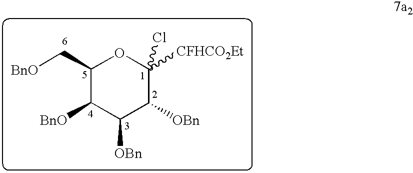

C₃₈H₄₀ClFO₇ M=663.17 g/mol

¹⁹F NMR (CDCl₃, 282 MHz) −194.9 (d, 1F, 46 Hz)

¹H NMR (CDCl₃, 300 MHz)

0.98 (t, 7.1 Hz, 3H, CH₃); 3.50 (dd, 5.4-9.1, 1H, H6) 3.58 (tapp., 8.6 Hz, 1H, H6); 3.92-4.09 (m, 4H, H4, H3, CH₂); 4.16 (m, 1H, H5); 4.37 (d, 11.8 Hz, 1H, OCH₂Ph); 4.43 (d, 11.4 Hz, 1H, OCH₂Ph); 4.46 (d, 10.9 Hz, 1H, OCH₂Ph); 4.48 (d, 9.3 Hz, 1H, H2); 4.63 (d, 11.4 Hz, 1H, OCH₂Ph); 4.68 (d, 11.4 Hz, 1H, OCH₂Ph); 4.75 (d, 11.5 Hz, 1H OCH₂Ph); 4.86 (d, 11.1 Hz, 1H, OCH₂Ph); 4.99 (d, 11.4 Hz, 1H, OCH₂Ph); 5.02 (d, 46 Hz, 1H, CHF); 7.23 (m, 20H, H ar.)

¹³C NMR (CDCl₃, 75.5 MHz)

14.2 (CH₃); 62.6 (CH₂); 67.6 (C6); 73.0 (OCH₂Ph); 73.8; 74.0 (OCH₂Ph) 74.4; 75.2 (20CH₂Ph); 76.0; 81.3; 89.2 (d, 200 Hz; CFH); 127.7-128.9 (C ar.); 138.3; 138.6; 139.0 (C quat. ar.); 161.4 and 161.9 (2t, 32 Hz, CO₂Et).

Synthesis of Compound 7b₁/7b₂ (FIG. 6)

The mixture of both diastereoisomers of ester 2b₁/2b₂ (500 mg; 0.77 mmol; 1 eq) is placed in dichloromethane (20 ml) and thionyl halide (138 mg; 1.16 mmol; 1.5 eq) is added at −30° C. After 30 min at −30° C., pyridine (92 mg; 1.16 mmol; 1.5 eq) is added and the solution is stirred for an additional 30 min. The solution is hydrolyzed with 2 N HCl (20 ml) and then extracted with dichloromethane (3×20 ml). The organic phases are washed with a saturated sodium chloride solution (30 ml) and then dried on sodium sulfate and concentrated. The crude reaction product is then purified by column chromatography on a silica gel with an eluent of cyclohexane/ethyl acetate (80:20).

The two diastereoisomers 2b₁/2b₂ are isolated in the form of a colorless oil with a yield of 78%.

Characterization of Compound 7b₁/7b₂

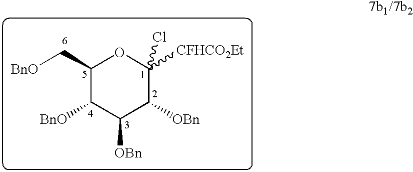

C₃₈H₄₀ClFO₇ M=663.17 g/mol

Rf=0.50, eluent: cyclohexane/ethyl acetate (8:2).

¹⁹F NMR (CDCl₃, 282 MHz)

−186.4 (1F, d, ²J_{F-H7} 47.2) 7b₁

−195.0 (1F, d, ²J_{F-H7} 46.1) 7b₂

¹H NMR (CDCl₃, 300 MHz)

1.10 (t, 3H, ³J_{H10-H10} 7.2, CH₃), 1.11-1.21 (m, 3H, CH₃), 3.55-3.78 (m, 3H, H6), 3.91-4.08 (m, 3H), 4.19 (q, 2H, ³J_{H9-H10} 7.2, CH₃), 4.39-4.57 (m, 4H), 4.67-5.20 (m, 5H, H7), 7.20-7.25 (20HAr).

¹³C NMR (CDCl₃, 75 MHz)

14.4 (CH₃), 14.5 (CH₃), 62.4 (CH₃), 62.8 (CH₃), 67.9 (C6), 68.2 (C6), 73.7, 73.9, 74.7, 75.4, 76.3, 79.3, 79.6, 83.0, 83.5, 88.9 (d, ¹J_{C7-F} 201.0, CF), 89.2 (d, ¹J_{C7-F} 195.9, CF), 103.0 (d, 2J_{C1-F} 22.8, Cl), 105.0 (d, ²J_{C1-F} 18.3, Cl), 127.3, 127.7, 127.7, 127.8, 127.8, 127.8, 127.9, 127.9, 128.0, 128.0, 128.1, 128.1, 128.3, 128.4, 128.5, 128.5, 128.6, 137.8, 137.9, 138.0, 138.1, 138.2, 138.3, 138.3, 138.3, 165.1 (d, ²J_{C8-F} 33.1, CO₂Et), 165.5 (d, 2J_{C8-F} 33.1, CO₂Et)

Synthesis of Compound 8b₁/8b₂ (FIG. 7)

Chlorinated product 7b₁/7b₂ (240 mg; 0.36 mmol; 1.0 eq) is placed with tributyltin (422 mg; 1.50 mmol; 4.0 eq) in dry toluene (20 ml) and the solution is refluxed for four hours. After returning to room temperature, the mixture is concentrated and purified by column chromatography on a silica gel with an eluent of cyclohexane/ethyl acetate (80:20). The product is isolated with a 63% yield.

The reaction can be carried out under the same conditions from brominated derivatives 6b₁/6b₂ to yield the same compounds 8b₁/8b₂.

Characterization of Compound 8b₁/8b₂

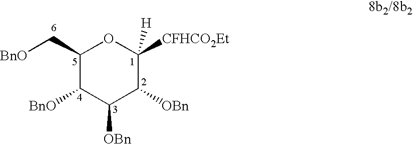

C₃₈H₄₁FO₇ M=628 g/mol

Rf=0.43, eluent: cyclohexane/ethyl acetate (8:2)

¹⁹F NMR (CDCl₃, 282 MHz)

−203.1 (1F, dd, ²J_{F-H7} 48.7, ³J_{F-H1} 23.6) 8b₂

−208.4 (1F, dd, ²J_{F-H7} 48.0, ³J_{F-H1} 31.1) 8b₁

¹H NMR (CDCl₃, 300 MHz)

1.03 (t, 3H, ³J_{H10-H9} 7.2, CH₃), 1.15 (t, 3H, ³J_{H10-H9} 6.7, CH₃), 3.50-3.72 (m, 7H, H2, H3, H4, H5 2H6), 3.88 (qₐₚₚ, 2H, ³J_{H9-H10} 6.7, CH₂), 4.15 (dd, 2H, ³J_{H9-H10} 7.2, 3.2, CH₂), 4.45-4.64 (m, 4H), 4.72-4.88 (m, 5H), 5.08 (d, 1H, ²J_{H7-F} 48.0, CFH), 5.08 (d, 1H, ²J_{H7-F} 48.7, CFH) 7.10-7.28 (m, 20H, H ar.).

¹³C NMR (CDCl₃, 75 MHz)

14.4 (CH₃), 14.6 (C10), 61.6 (CH₃), 61.8 (CH₂), 68.9 (C6), 73.8, 74.0, 75.0, 75.6, 75.7, 76.1, 76.8 (d, J^{C-F} 7.0), 77.5 (d, J_{C-F} 4.0), 78.6, 78.8, 79.4, 79.6, 79.9, 80.5, 87.0 (d, ¹J_{C7-F} 191.8, CF), 87.5 (d, ²J_{C1-F} 22.1, Cl), 88.6 (d, ¹J_{C7-F} 190.8, CF), 127.6, 127.6, 127.7, 127.8, 127.8, 127.9, 127.9, 128.0, 128.0, 128.1, 128.2, 128.2, 128.3, 128.4, 128.5, 128.6, 128.6, 128.7, 137.9, 138.0, 138.2, 138.2, 138.3, 167.0 (d, ²J_{C8-F} 24.6, CO₂Et), 167.7 (d, ²J_{C8-F} 24.6, CO₂Et).

Synthesis of Compound 9b₁/9b₂ (FIG. 8)

In a flask under inert atmosphere containing ester 8b₁/8b₂ (140 mg; 0.223 mmol; 1 eq) in solution in ethanol (12 ml), an aqueous lithium hydroxide solution (2 M; 2 eq) is added and the mixture is stirred overnight at room temperature. The mixture is concentrated and dissolved in DCM (15 ml) and then acidified with a 2 M HCl solution (20 ml). The mixture is extracted with DCM (3×20 ml) and the organic phases are combined, washed with a saturated NaCl solution (30 ml) and concentrated directly. Acid 9b₁/9b₂ is isolated as a colorless oil with a crude yield of 99%.

Characterization of Compound 9b₁/9b₂

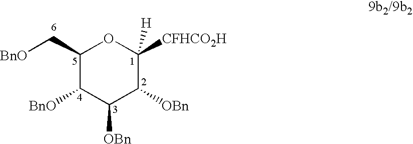

C₃₆H₃₇FO₇ M=600 g/mol

¹⁹F NMR (CDCl₃, 282 MHz)

−202.1 (1F, dd, ²J_{F-H7} 45.1, ³J_{F-H1} 19.3) 9b₂

−207.2 (1F, dd, ²J_{F-H7} 46.1, ³J_{F-H1} 30.0) 9b₁

¹H NMR (CDCl₃, 300 MHz)

3.46-3.72 (m, 7H, H1, H2, H3, H4, H5, 2H6), 4.43-4.50 (m, 2H), 4.54-4.87 (m, 6H), 5.02 (d, ²J_{H7-F} 47.8, CFH), 5.06 (d, ²J_{H7-F} 48.5, CFH), 7.09-7.31 (20H ar.).

¹³C NMR (CDCl₃, 75 MHz)

68.6, 68.8, 73.3, 73.6, 74.8, 75.3, 75.4, 75.8, 76.4, 76.5, 77.5, 77.5, 78.2, 78.7, 79.0, 79.5, 79.7, 86.3 (d, ¹J_{C7-F} 191.0, CF), 86.6 (d, ²J_{C1-F} 18.8, C1), 87.7 (d, ¹J_{C7-F} 191.9, C7), 127.5, 127.6, 127.8, 127.8, 127.9, 128.0, 128.1, 128.1, 128.2, 128.2, 128.4, 128.5, 128.6, 128.6, 128.7, 137.8, 137.9, 138.3, 138.4, 171.5 (d, ²J_{C8-F} 24.6 CO₂Et).

Synthesis of Compound 10b₂ (FIG. 9)

In a flask under inert atmosphere containing ester 2b₂ (290 mg; 0.45 mmol; 1 eq) in solution in anhydrous toluene (5 ml) at −78° C., a solution of 1 M DIBAL-H in toluene is added (1.35 ml; 1.35 mmol; 3 eq). The mixture is stirred at −78° C. for 2.5 hours and then another DIBAL-H solution is added (450 μl; 0.45 mmol; 1 eq). The mixture is stirred again for 30 min at −78° C. and then ethanol is added (2 ml). The solution is brought up to −20° C. over 10 min, and a 20% solution of Rochelle salts (40 ml) is added. The mixture is stirred rapidly for several hours. Ethyl acetate (20 ml) is added. After decanting, the aqueous phase is extracted twice with ethyl acetate (2×15 ml). Next, the organic phases are recombined and washed twice with a saturated aqueous NaCl solution (2×15 ml), dried on magnesium sulfate, filtered and then evaporated.

Characterization of 10b₂

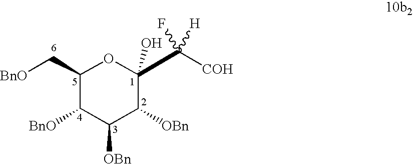

C₃₆H₃₇FO₇ M=600.67 g/mol

¹⁹F NMR (CDCl₃, 282.5 MHz)

−211.4 (dd, J_{F-H}=48 Hz and 8 Hz)

¹H NMR (CDCl₃, 300 MHz) 3.57-3.62 (m, 4H, H6, H2, H4); 3.96 (m, 1H, H5); 3.97 (t, 9.1 Hz, 1H, H3); 4.44 (d, 12.3 Hz, 1H, OCH₂Ph); 4.48 (d, 12.3 Hz, 1H, OCH2ₚh); 4.50 (d, 11.8 Hz, 1H, OCH₂Ph); 4.53 (d, 10.8 Hz, 1H, OCH₂Ph); 4.6 (d, 49 Hz, 1H, CHF); 4.72 (d, 10.8 Hz, 1H, OCH₂Ph); 4.77 (d, 11.2 Hz, 1H, OCH₂Ph); 4.85 (d, 11.1 Hz, 1H, OCH₂Ph); 7.07-7.25 (m, 20H, H ar.); 9.45 (d, 7.4 Hz, 1H, CHO).

¹³C NMR (CDCl₃, 75.5 MHz)

68.8 (C6); 72.6 (C5); 73.8 (OCH₂Ph); 75.4 (OCH₂Ph); 75.5 (OCH₂Ph); 76.0 (OCH₂Ph); 77.9 (C2); 78.4 (C4); 83.5 (C3); 94.1 (d, 192 Hz, CHF); 98.2 (d, 20 Hz, Cl); 128.0-128.9 (C ar.); 137.6; 138.3; 138.4; 138.6 (C quat. ar.); 196.2 (d, 30 Hz, CHO).

Synthesis of Compound 10a₁ (FIG. 11)

In a flask under inert atmosphere at −78° C. containing DMSO (12 μl; 0.16 mmol; 5 eq) in dichloromethane (1 ml), a solution of oxalyl chloride (7 μl, 0.073 mmol; 2.2 eq) in dichloromethane (1 ml) is added. The reaction mixture is stirred for 15 min at −78° C., then alcohol 11a₁ (20 mg; 0.033 mmol; 1 eq) is added. The temperature is brought up to −40° C. over 1 hour and then triethylamine (24 μl; 0.17 mmol; 5 eq) is introduced. The temperature is allowed to return to room temperature over two hours, and then a saturated NaCl solution is added. The mixture is extracted three times with dichloromethane, and then the organic phases are recombined and washed with an aqueous solution, dried on magnesium sulfate, filtered and then concentrated. The product is then purified by column chromatography on a silica gel with an eluent of cyclohexane/ethyl acetate (80:20). The product is isolated with a 31% yield in the form of a colorless oil.

Characterization of 10a₁

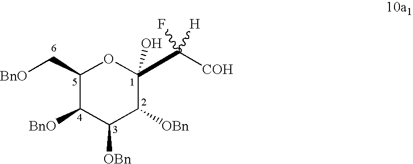

C₃₆H₃₇FO₇ M=600.67 g/mol

Rf=0.45, eluent: cyclohexane/ethyl acetate (6:4)

¹⁹F NMR (CDCl₃, 282.5 MHz) −205.4 (dd, J_{F-H}=47.3 Hz and 7.5 Hz)

¹H NMR (CDCl₃, 300 MHz)

3.51 (dd, 11.4 Hz and 1.7 Hz, 1H, 1H6); 3.63-3.77 (m, 3H, 1H6, H2; 3.88-3.97 (m, 2H, H5, H3); 4.39-4.9 (m, 8H, 4OCH₂Ph); 4.62 (d, 47.7 Hz, 1H, CHF); 7.07-725 (m, 20H, H ar.); 974 (d, 7 Hz, 1H, CHO).

¹³C NMR (CDCl₃, 75.5 MHz)

688 (C6); 730 (C5); 73 (OCH₂Ph); 755 (OCH₂Ph); 760 (OCH₂Ph); 761 (OCH₂Ph); 78.2 (C2); 78.4 (C4); 83.2 (C3); 91.1 (d, 175 Hz, CHF); 128.0-129.0 (C ar.); 137.8; 138.3; 138.4; 138.7 (C quat. ar.); 200 (d, 30 Hz, CHO).

Mass (ESI+): 601 (M+H⁺); 618.27 (M: hydrate H₂O); 636.27 (M: hydrate 2H₂O).

Synthesis of Compound 10a₂ (FIG. 14)

In a flask under inert atmosphere at −78° C. containing product 13a2 (0.165 mmol; 1 eq) in anhydrous tetrahydrofuran (3 ml), a solution of 1 M DIBAL-H in toluene (0.25 ml, 0.25 mmol, 1.5 eq) is added slowly. The mixture is stirred at −78° C. for 4 hours. Next, a saturated NH₄Cl solution is added and the mixture is returned to room temperature. After adding a 1 M HCl solution, the mixture is extracted three times in dichloromethane. The organic phases are then recombined and washed with a saturated NaHCO₃ solution, dried on magnesium sulfate, filtered and then concentrated. The product is then purified by column chromatography on a silica gel with an eluent of cyclohexane/ethyl acetate (80:20). The product is isolated with a 30% yield in the form of a colorless oil.

Characterization of 10a₂

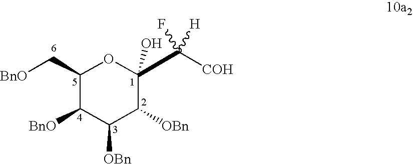

C₃₆H₃₇FO₇ M=600.67 g/mol

Rf=0.6, eluent: cyclohexane/ethyl acetate (7:3)

¹⁹F NMR (CDCl₃, 282.5 MHz)

−211.1 (dd, J_{F-H}=47.2 Hz and 7.5 Hz)

¹H NMR (CDCl₃, 300 MHz) 3.55-3.46 (m, 2H, H6); 3.8 (s, 1H, OH); 3.90 (dd, 9.7 Hz and 2.7 Hz, 1H, H3); 3.92 (s, 1H, H4); 4.06 (t, 9.6 Hz, 1H, H5,); 4.12 (d, 9, 0.6 Hz, 1H, H2,); 4.34 (d, 11.9 Hz, 1H, OCH₂Ph); 4.39 (d, 11.9 Hz, 1H, OCH₂Ph); 4.51 (d, 11.8 Hz, 1H, OCH₂Ph); 4.52 (d, 10.6 Hz, 1H, OCH₂Ph); 4.57 (d, 11.6 Hz, 1H, OCH₂Ph); 4.59 (d, 47.6 Hz, 1H, CHF); 4.67 (d, 11.5 Hz, 1H, OCH₂Ph); 4.85 (d, 11.8 Hz, 1H, OCH₂Ph); 4.88 (d, 10.6 Hz, 1H, OCH₂Ph); 7.30 (m, 20H, H ar.); 9.41 (d, 8.3 Hz, 1H, CHO).

¹³C NMR (CDCl₃, 75.5 MHz)

68.8 (C6); 71.4 (C5); 72.7 (OCH₂Ph); 73.9 (OCH₂Ph); 74.0 (C4); 74.5 (C2); 74.7 (OCH₂Ph); 75.4 (OCH₂Ph); 81.1 (C3); 94.4 (d, 190.8 Hz, CHF); 98.6 (d, 20 Hz, Cl); 127.9; 128.0; 128.1; 128.2; 128.3; 128.4; 128.7; 128.8; 128.9; 129.0 (C ar.); 137.8; 138.1; 138.3; 139.0 (C quat. ar.); 195.5 (d, 28 Hz, CHO).

Mass (ESI+): 655.33 (M: hydrate OMe+Na).

Synthesis of Compound 11a₁ (FIG. 10)

In a flask under inert atmosphere containing ester 2a₁ (52 mg; 0.08 mmol; 1 eq) in solution in ethanol (5 ml), NaBH₄ (10 mg, 0.241 mmol, 3 eq) is added and the mixture is stirred for 12 hours. The mixture is concentrated and then taken up in a mixture of dichloromethane and water. After decanting, the aqueous phase is extracted three times in dichloromethane. Next, the organic phases are recombined and washed with a saturated aqueous NaCl solution (15 ml), dried on magnesium sulfate, filtered and then evaporated. The crude product is then purified by column chromatography on a silica gel with an eluent of cyclohexane/ethyl:acetate (50:50). The product is isolated with a 38% yield in the form of a colorless oil.

Characterization of 11a₁

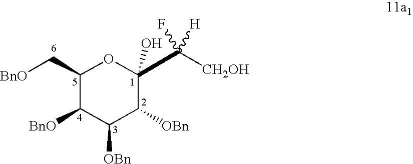

C₃₆H₃₉FO₇ M=602.69 g/mol

Rf=0.53 (cyclohexane/ethyl acetate 5/5)

¹⁹F NMR (CDCl₃, 282.5 MHz)

−199.2 (dt, J_{F-H}=46.1 Hz and 19.3 Hz)

¹H NMR (CDCl₃, 300 MHz) 2.11 (s, 1H, OH); 3.58-3.65 (m, 3H, H2 and 2H6); 3.81-4.00 (m, 5H, CH₂, H4, H5H3; 4.44 (td, 3.7 Hz and 46.5 Hz, 1H, CHF); 4.48-4.81 (m, 8H, 4 OCH₂Ph); 7.26 (m, 20H, Har.).

¹³C NMR (CDCl₃, 75.5 MHz)

60.4 (d, 24.6 Hz, CH₂); 68.8 (C6); 72.1 (C5; 73.6 (OCH₂Ph); 75.2 (OCH₂Ph); 75.9 (2C) (OCH₂Ph); 78.1 (C2); 78.6 (C4); 83.3 (Cl); 90.1 (d, 183 Hz, CHF); 97.9 (d, 22 Hz, Cl); 127.8; 127.9 (2C); 128.0; 128.1; 128.3; 128.6 (2C) (C ar.); 137.8; 138.0 (2C); 138.4 (C quat. ar.).

Mass (ESI+): 625.33 (M+Na).

Synthesis of Compound 11b₂ (FIG. 10)

In a flask under inert atmosphere containing ester

2b₂ (52 mg; 0.08 mmol; 1 eq) in solution in ethanol (5 ml), NaBH₄ (10 mg, 0.241 mmol, 3 eq) is added and the mixture is stirred for 12 hours. The mixture is concentrated and then taken up in a mixture of dichloromethane and water. After decanting, the aqueous phase is extracted three times in dichloromethane. Next, the organic phases are recombined and washed with a saturated aqueous NaCl solution (15 ml), dried on magnesium sulfate, filtered and then evaporated. The crude product is then purified by column chromatography on a silica gel with an eluent of cyclohexane/ethyl:acetate (50:50). The product is isolated with a 42% yield in the form of a colorless oil.

Characterization of 11b₂

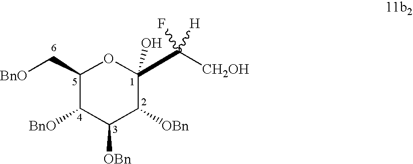

C₃₆H₃₉FO₇ M=602.69 g/mol

Rf=0.53 (cyclohexane/ethyl acetate 5/5),

¹⁹F NMR (CDCl₃, 282.5 MHz)

−209.7 (dddd, J_{F-H}=47.3 Hz, 27 Hz, 22.6 Hz and 4.3 Hz)

¹H NMR (CDCl₃, 300 MHz)

2.26 (dd, 5.1 Hz and 7 Hz, 1H, OH); 3.44 (dd, 0.9 Hz and 9.1 Hz, 1H, H2); 3.50-3.67 (m, 5H, 2H6, CH₂, H4); 3.89-3.94 (ddd, 2.2 Hz, 3.2 Hz and 10 Hz, 1H, H5); 4.00 (t, 9.1 Hz, 1H, H3); 4.36 (td, 3.7 Hz and 46.5 Hz, 1H, CHF); 4.38 (d, 12 Hz, 1H, OCH₂Ph); 4.47 (d, 12 Hz, 1H, OCH₂Ph); 4.48 (d, 10.9 Hz, 1H, OCH₂Ph); 4.63 (d, 11.2 Hz, 1H OCH₂Ph); 4.74 (d, 11 Hz, 1H, OCH₂Ph); 4.82 (d, 10.1 Hz, 1H, OCH₂Ph); 4.86 (d, 11.2 Hz, 1H, OCH₂Ph); 7.09-7.12 (m, 20H, H ar.); 9.45 (d, 7.4 Hz, 1H, CHO).

¹³C NMR (CDCl₃, 75.5 MHz)

61.2 (d, 22 Hz, CH₂); 68.5 (C6) 71.8 (C5); 73.8 (OCH₂Ph); 75.4 (OCH₂Ph); 76.1 (OCH₂Ph); 77.6 (OCH₂Ph); 78.1 (C2 or C4); 79.2 (C2 or C4); 83.5 (C3); 93.2 (d, 179 Hz, CHF); 97.6 (d, 20 Hz, Cl); 128.1; 128.2 (2C); 128.3; 128.7; 128.8; 128.9; 129.0; 129.3 (C ar.); 137.7; 138.3; 138.4; 138.7 (C quat. ar.).

Synthesis of Compound 12a₁ (FIG. 12)

In a flask under inert atmosphere containing alcohol 11a₁ (34 mg; 0.056 mmol; 1 eq) in solution in anhydrous dichloromethane (1 ml) at 0° C., triethylamine (10 μl; 0.075 mmol; 1.3 eq) and then MsCl (7 μl, 0.075 mmol, 1.3 eq) are slowly added. The mixture is stirred at 0° C. for 30 min and then at room temperature for 3 hours. The mixture is hydrolyzed and then extracted three times in dichloromethane. Next, the organic phases are recombined and washed with a saturated aqueous NaCl solution, dried on magnesium sulfate, filtered and then evaporated. The crude product is then purified by column chromatography on a silica gel with an eluent of cyclohexane/ethyl acetate (70:30). The product is isolated with a 50% yield in the form of a white oil.

Characterization of 12a₁

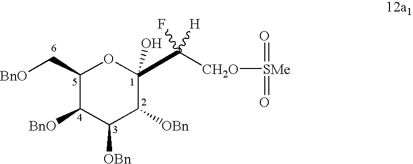

C₃₇H₄₁FO₉S M=680.8 g/mol

Rf=0.83 (cyclohexane/ethyl acetate 5/5)

¹⁹F NMR (CDCl₃, 282.5 MHz)

−186.3 (ddd, J_{F-H}=51.5 Hz, 35.4 Hz and 18.2 Hz)

¹H NMR (CDCl₃, 300 MHz)

2.95 (s, 3H, CH₃); 3.45 (dd, 5.6 Hz and 9.1Hz, 1H, 1H6); 3.55 (dd, 7.6 Hz and 9.1 Hz, 1H, 1H6); 3.98 (1s, 2H, H3; 4.08 (t, 6.8 Hz, 1H, H5); 4.37 (is, 3H, H2 and OCH₂Ph); 4.54 (d, 11.5 Hz, 1H, OCH₂Ph); 4.67 (s, 2H, OCH₂Ph); 4.68 (d, 1H, OCH₂Ph); 4.73 (dd, 30 Hz and 12.3 Hz, 2H, CH₂); 4.9 (d, 11.4 Hz, OCH₂Ph); 4.93 (d, 11.3 Hz, 1H, OCH₂Ph); 4.97 (dd, 8.3 Hz and 48 Hz, 1H, CHF); 7.24 (m, 20H, H ar.).

¹³C NMR (CDCl₃, 75.5 MHz)

37.8 (CH₃); 67.7 (C); 68.3 (d, 21.7 Hz, CH₂); 73.1 (OCH₂Ph); 73.6 (C4); 73.7 (OCH₂Ph); 74.9 (d, 3.5 Hz, C2); 75.0 (OCH₂Ph); 75.1 (C5); 75.7 (OCH₂Ph); 80.3 (C3); 90.5 (d, 189.6 Hz, CHF); 106.0 (d, 21.1 Hz; 127.7; 127.8; 128.0; 128.1 (2C); 128.5; 128.6; 128.7 (2C) (C ar.); 137.7; 137.9; 138.1; 138.5 (C quat. ar.).

Mass (ESI+): 680 (M+H⁺); 716 (M: hydrate 2H₂O)

Synthesis of Compound 13a₂ (FIG. 13)

In a flask under inert atmosphere at −0° C. containing 1 M Me₂AlCl in cyclohexane (1.5 ml; 1.44 mmol, 3 eq) in dichloromethane (15 ml), Weinreb amine (141 mg; 1.44 mmol; 3 eq) is added and then the mixture is stirred for one hour. Ester 2a₂ (311 mg; 0.481 mmol; 1 eq) in anhydrous dichloromethane (5 ml) is added at 0° C. and then the solution is allowed to rise to room temperature with stirring over 12 hours. The reaction is hydrolyzed with a 1 M HCl solution, filtered on celite, dried on magnesium sulfate and then concentrated. The product is then purified by column chromatography on a silica gel with an eluent of cyclohexane/ethyl acetate (50:50). The product is isolated with a 75% yield in the form of a yellow oil.

Characterization of 13a₂

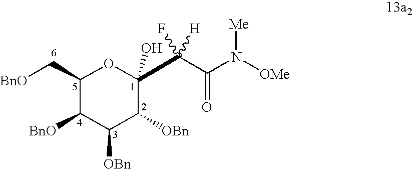

C₃₈H₄₂FO₈ M=659.76 g/mol

Rf=0.62, eluent: cyclohexane/ethyl acetate (5:5)

¹⁹F NMR (CDCl₃, 282.5 MHz)

−196.8 (d, J_{F-H}=48.3 Hz)

¹H NMR (CDCl₃, 300 MHz)

3.1 (s, 3H, CH₃); 3.44-3.57 (m, 2H, H6); 3.59 (s, 3H, OCH₃); 3.93 (s, 1H, H4); 4.00-4.04 (m, 2H, H2, H3); 4.15 (t, 9.6 Hz, 1H, H5,); 4.37 (m, 2H, OCH₂Ph); 4.53 (d, 11.5 Hz, 1H, OCH₂Ph); 4.64-4.78 (m, 3H, OCH₂Ph); 4.84-4.94 (m, 2H, OCH₂Ph); 5.14 (d, 48.2 Hz, 1H, CHF); 7.21-7.29 (m, 20H, H ar.).

¹³C NMR (CDCl₃, 75.5 MHz)

32.3 (CH₃); 62.3 (OCH₃); 68, (C6); 70.5 (CS); 73.4 (OCH₂Ph); 73.7 (OCH₂Ph); 75.0 (OCH₂Ph); 75.2 (C4); 75.6 (OCH₂Ph); 80.7 (C2 and C3); 127.9; 128.0; 128.2 (2C); 128.6 (2C); 128.8; 128.9; 129.1 (C ar.); 138.4; 138.7; 138.9; 139.4 (C quat. ar.).

Mass (ESI+): 660 (M+H); 677 (M+H₂O)

Synthesis of Compounds 14a₁/14a₂ (FIG. 15)

To a suspension of acid 2a₁/2a₂ (253 mg; 0.42 mmol; 1.0 eq), alanine (0.42 mmol; 1.0 eq), HOBt (65 mg; 0.47 mmol; 1.1 eq), and NMM (143 mg; 1.41 mmol; 3.3 eq) in DMF (15 ml) under an argon atmosphere, EDCI (90 mg; 0.47 mmol; 1.1 eq) is added after 15 minutes. The reaction is stirred at room temperature for 24 hours and then concentrated. A 1 N hydrochloric acid solution (10 ml) is added as well as dichloromethane, and the aqueous phase is extracted with DCM (3×20 ml). The organic phases are washed with a saturated NaCl solution (20 ml), dried on MgSO₄ and vacuum concentrated. The residue is purified by chromatography on a silica gel with as eluent a mixture of cyclohexane/AcOEt (70:30) to allow separation of the two diastereoisomers 14a₁ and 14a₂ in the form of two white solids with a total yield of 60%.

Characterization of 14a₁/14a₂

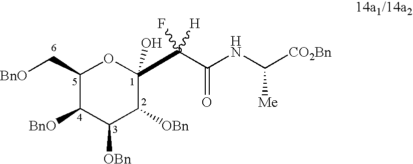

C₄₆H₄₈FNO₉ M=777.87 g/mol

Rf=, eluent: cyclohexane/ethyl acetate ( ).

¹⁹F NMR (CDCl₃, 282.5 MHz)

−200 (d, J_{F-H}=47.3 Hz)

¹H NMR (CDCl₃, 300 MHz)

1.25 (d, 7.2 Hz, 3H, CH₃); 3.43 (dd, 5.6 Hz and 9.4 Hz, 1H, 1H6); 3.59 (dd, 7.6 Hz and 9.4 Hz, 1H, 1H6); 4.01 (s, 1H, H4); 4.11 (dd, 2.6 Hz and 10 Hz, 1H, H3); 4.19 (t, 6.3 Hz, 1H, H5,); 4.24 (d, 10 Hz, 1H, H2); 4.37 (d, 11.7 Hz, 1H, OCH₂Ph); 4.43 (d, 11.7 Hz, 1H, OCH₂Ph); 4.62 (m, 1H, CH); 4.66 (d, 11.7 Hz, 1H, OCH₂Ph); 4.71 (d, 11.3 Hz, 1H, OCH₂Ph); 4.76 (s, 2H, OCH₂Ph); 4.56 (d, 11.7 Hz, 1H, OCH₂Ph); 4.99 (d, 47.7 Hz, 1H, CHF); 5.00 (d, 11.3 Hz, 1H, OCH₂Ph); 5.19 (s, 2H, CO₂CH₂Ph); 5.55 (s, 1H, OH); 7.07 (dd, 3.5 Hz and 7.6 Hz, 1H, NH); 7.21-7.29 (m, 25H, H ar.).

¹³C NMR (CDCl₃, 75.5 MHz)

18.3 (CH₃); 48.1 (CH); 67.5 (CO₂ CH₂Ph) 68.8 (C6); 70.6 (C5); 72.9 (OCH₂Ph); 73.5 (OCH₂Ph); 74.4 and 74.5 (C2 and C4); 74.6 (OCH₂Ph); 75.6 (OCH₂Ph); 80.4 (C3); 86.6 (d, 201.8 Hz, CFH); 98.1 (d, 20.7 Hz, Cl); 127.6; 127.9; 128.3; 128.4; 128.5; 128.6; 128.8 (C ar.); 135.2; 138.0; 138.1; 138.5; 138.9 (C quat. ar.); 168.9 (d, 19.6 Hz, COCFH); 171.8 (CO).

Mass (ESI+): 760.27 (M+H); 795.2 (M+H₂O)

### Compound 14a2

Rf=, eluent: cyclohexane/ethyl acetate ( ).

¹⁹F NMR (CDCl₃, 282.5 MHz)

−201.9 (d, J_{F-H}=47.3 Hz)

¹H NMR (CDCl₃, 300 MHz) 1.23 (d, 7.0 Hz, 3H, CH₃); 3.49 (dd, 5.8 Hz and 9.1 Hz,

¹H, 1H6); 3.56 (dd, 7.5 Hz and 9.1 Hz, 1H, 1H6); 3.94 (s, 1H, H4); 4.0 (dd, 2.7 Hz and 9.7 Hz, 1H, H3); 4.12-4.23 (m, 2H, H5 and H2); 4.35 (d, 11.8 Hz, 1H, OCH₂Ph); 4.42 (d, 11.8 Hz, 1H, OCH₂Ph); 4.49-5.10 (m, 10H, CH; 40CH₂Ph; CHF); 7.09-7.24 (m, 25H, H ar.)

Synthesis of Compound 15a₁ (FIG. 16)

In a flask, compound 14a₁ (0.100 mmol) is dissolved in tetrahydrofuran (10 ml) with water (5 ml) and palladium on carbon and then placed under an atmosphere of hydrogen. The mixture is stirred for two days at room temperature. The reaction mixture is filtered and then concentrated. The crude product is taken up in dichloromethane (20 ml), which is eliminated, and then in water (10 ml), which is filtered. The aqueous phase is then concentrated thus leaving the desired product as a pale yellow solid with a yield of 98%.

Characterization of 15a₁

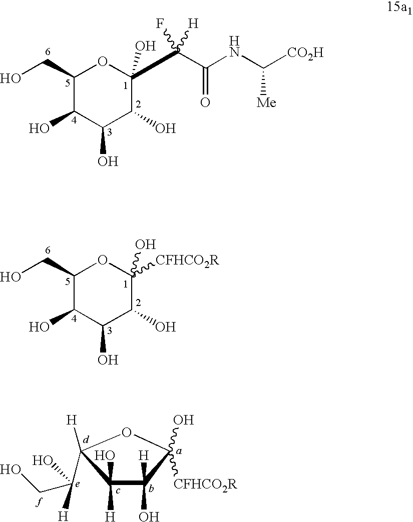

C₁₁H₁₈FNO₉ M=327.26 g/mol

¹H NMR (D₂O, 300 MHz)

1.4 (d, 7.2 Hz, 3H, CH₃); 3.6-3.0 (m, H6, He, Hf); 3.95 (s, H4); 4 (m, H2 and H3); 4.05 (t, 1H, H5); 4.1-4.3 (m, Hb, Hc and Hd); 4.5 (m, 1H, CH); 5.1 (2D, 47 HZ, CHF)

¹³C NMR (D₃O, 75.5 MHz)

15.0 (CH₃); 47.4 (CH); 59.8 (C6); 65.9 (C4); 68.1 and 68.9 (C2 and C3); 70.9 (C5); 86.6 (d, 198 Hz, CFH); 96.4 (d, 20.7 Hz, Cl); 170 (d, 19.6 Hz, COCFH); 171.8 (CO).

Mass (ESI+): 345.03 (M+Na)

Synthesis of Compound 16a₁ (FIG. 17)

To a suspension of acid 5a₁ (253 mg; 0.42 mmol; 1.0 eq), peptide (0.42 mmol; 1.0 eq), HOBt (65 mg; 0.47 mmol; 1.1 eq), and NMM (143 mg; 1.41 mmol; 3.3 eq) in DMF (15 ml) under an argon atmosphere, EDCI (90 mg; 0.47 mmol; 1.1 eq) is added after 15 minutes. The reaction is stirred at room temperature for 24 hours and then concentrated. A 1 N hydrochloric acid solution (10 ml) is added as well as dichloromethane, and the aqueous phase is extracted with DCM (3×20 ml). The organic phases are washed with a saturated NaCl solution (20 ml), dried on MgSO₄ and vacuum concentrated. The residue is purified by chromatography on a silica gel with as eluent a mixture of cyclohexane/AcOEt (30:70) to isolate compound 16a₁ in the form of a white solid with a total yield of 56%.

Characterization of 16a₁

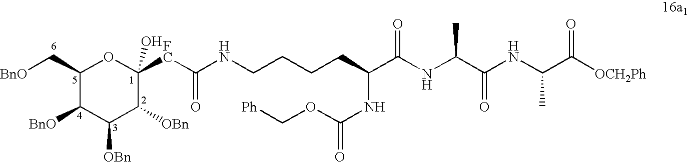

C₆₃H₇₀FN₄O₁₃ M=1110.25 g/mol

Rf=, eluent: cyclohexane/ethyl acetate ( ).

¹⁹F NMR (CDCl₃, 282.5 MHz)

−199.4 (d, J_{F-H}=48.4 Hz)

¹H NMR (CDCl₃, 300 MHz)

1.22 and 1.30 (2D, 7. Hz, 6H, 2CH₃); 1.22-1.51 (m, 3CH₂lys); 3.01-3.07 (m, 1H, 1CH₂NH); 3.23-3.30 (m, 1H, 1CH₂NH) 3.37 (dd, 5.8 Hz and 9.4 Hz, 1H, 1H6); 3.47 (dd, 9.2 Hz, 1H, 1H6); 4.01 (s, 1H, H4); 4.1 (dd, 1.8 Hz and 10 Hz, 1H, H3); 4.15-4.24 (m, 3H, H5, CHLys, H2); 4.45-4.73 (m, 6H, 2CHAla, OCH2Ph); 4.77 (s, 2H, OCH2Ph); 4.93-5.21 (m, 6H, OCH₂Ph); 5.01 (d, 47.2 Hz, 1H, CHF); 5.51 (d, 6.9 Hz, 1H, NH); 5.65 (s, 1H, OH); 6.49 (d, 7.4 Hz, 1H, NH); 6.62 (d, 7.3 Hz, 1H, NH); 6.73 (s, 1H, NH); 7.18-7.23 (m, 25H, H ar.).

¹³C NMR (CDCl₃, 75.5 MHz)

18.1 and 18.3 (2CH₃); 22.3 (CH₂); 28.7 (CH₂); 32.0 (CH₂); 38.5 (NCH₂); 48.3 and 48.9 (2CHAla); 54.8 (CHLys); 67.1 (OCH2Ph); 67.2 (OCH2Ph); 68.5 (C6); 70.4 (C5); 72.8 (OCH₂Ph); 73.4 (OCH₂Ph); 74.6 (C4 and C2); 74.7 (OCH₂Ph); 75.5 (OCH₂Ph); 80.3 (C3); 86.8 (d, 200.7 Hz, CFH); 98.1 (d, 20.7 Hz, C1); 127.0; 127.6; 128.0; 128.2; 128.3 (2C); 128.4 (2C); 128.5; 128.6; 128.7 (C ar.); 136.3; 138.0; 138.2; 138.3; 138.5; 138.7; 138.8; 138.9 (C quat. ar.); 156.0 (CO); 169.4 (d, 19 Hz, COCFH); 171.7 and 172.6 (2CO).

Mass (ESI+): 1128.33 (M+H₂O)

Synthesis of Compound 17a₁ (FIG. 18)

In a flask, compound 16a₁ (0.028 mmol) is dissolved in tetrahydrofuran (5 ml) with a 1 N hydrochloric acid solution (1.2 eq) and palladium on carbon and placed under an atmosphere of hydrogen. The mixture is stirred for two days at room temperature. The reaction mixture is filtered and then concentrated. The crude product is taken up in dichloromethane (20 ml), which is eliminated, and then in water (10 ml), which is filtered. The aqueous phase is then concentrated thus leaving the desired product as a white solid with a quantitative yield.

Characterization of 17a₁

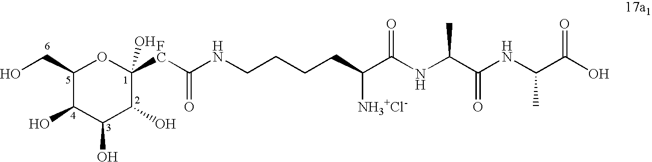

C₂₀H₃₅ClFN₄O₁₁ M=561.96 g/mol

¹⁹F NMR (CDCl₃, 282.5 MHz)

−199.4 (d, J_{F-H}=48.4 Hz)

¹H NMR (CDCl₃, 300 MHz)

1.22 and 1.30 (2D, 7, Hz, 6H, 2CH₃); 1.22-1.51 (m, 6H, 3CH₂lys); 3.01-3.07 (m, 1H, 1CH₂NH); 3.23-3.30 (m, 1H, 1CH₂NH) 3.37 (dd, 5.8 Hz and 9.4 Hz, 1H, 1H6); 3.47 (dd, 9.2 Hz, 1H, 1H6); 4.01 (s, 1H, H4); 4.1 (dd, 1.8 Hz and 10 Hz, 1H, 4.15-4.24 (m, 3H, H5, CHLys, H2); 4.45-4.73 (m, 6H, 2CHAla, OCH2Ph); 4.77 (s, 2H, OCH₂Ph); 4.93-5.21 (m, 6H, OCH₂Ph); 5.01 (d, 47.2 Hz, 1H, CHF); 5.51 (d, 6.9 Hz, 1H, NH); 5.65 (s, 1H, OH); 6.49 (d, 7.4 Hz, 1H, NH); 6.62 (d, 7.3 Hz, 1H, NH); 6.73 (s, 1H, NH); 7.18-7.23 (m, 25H, H ar.).

¹³C NMR (CDCl₃, 75.5 MHz)

18.1 and 18.3 (2CH₃); 22.3 (CH₂); 28.7 (CH2); 32.0 (CH₂); 38.5 (NCH₂); 48.3 and 48.9 (2CHAla); 54.8 (CHLys); 67.1 (OCH₂Ph); 67.2 (OCH₂Ph); 68.5 (C6); 70.4 (C5); 72.8 (OCH₂Ph); 73.4 (OCH₂Ph); 74.6 (C4 and C2); 74.7 (OCH₂Ph); 75.5 (OCH₂Ph); 80.3 (C3); 86.8 (d, 200.7 Hz, CFH); 98.1 (d, 20.7 Hz, C1); 127.0; 127.6; 128.0; 128.2; 128.3 (2C); 128.4 (2C); 128.5; 128.6; 128.7 (C ar.); 136.3; 138.0; 138.2; 138.3; 138.5; 138.7; 138.8; 138.9 (C quat. ar.); 156.0 (CO); 169.4 (d, 19 Hz, COCFH); 171.7 and 172.6 (2CO).

Mass (ESI+): 1128.33 (M+H₂O)

Synthesis of Compound 18b₁ (FIG. 19)

To a suspension of acid 5b₁ (500 mg; 0.811 mmol; 1.05 eq), 4-aminophenol (84 mg, 0.773 mmol; 1.0 eq), HOBt (110 mg; 0.811 mmol; 1.05 eq), and NMM (80 μl mg; 0.811 mmol; 1.05 eq) in DMF (20 ml) under an argon atmosphere, EDCI (156 mg; 0.811 mmol; 1.05 eq) is added after 15 minutes. The reaction is stirred at room temperature for 24 hours and then concentrated. A 1 N hydrochloric acid solution (10 ml) is added as well as dichloromethane, and the aqueous phase is extracted with DCM (3×20 ml). The organic phases are washed with a saturated NaCl solution (20 ml), dried on MgSO₄ and vacuum concentrated. The residue is purified by chromatography on a silica gel with as eluent a mixture of cyclohexane/AcOEt (60:40) to isolate compound 18b₁ in the form of a white solid with a total yield of 44%.

Characterization of 18b₁

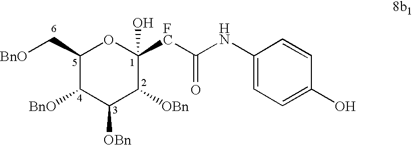

C₄₂H₄₁FNO₈ M=706.78 g/mol

Rf=0.27, eluent: cyclohexane/ethyl acetate (7/3)

¹⁹F NMR (CDCl₃, 282.5 MHz)

−197.6 (d, J_{F-H}=47 Hz)

¹H NMR (CDCl₃, 300 MHz) 3.53 (dd, 11.3 Hz, 1H, 1H6); 3.55-3.71 (m, 3H, 1H6, H4, H2); 3.98 (m, 1H, H5); 4.1 (t, 9.4 Hz, 1H, H3); 4.36 (d, 12.2 Hz, 1H, OCH₂Ph); 4.42 (d, 12.4 Hz, 1H, OCH₂Ph); 4.53 (d, 10.9 Hz, 1H, OCH₂Ph); 4.64 (d, 11.5 Hz, 1H, OCH₂Ph); 4.79 (d, 10.9 Hz, 1H, OCH₂Ph); 4.86 (s, 2H, OCH₂Ph); 4.92 (d, 11.4 Hz, 1H, OCH₂Ph); 4.95 (d, 48 Hz, 1H, CHF) 5.46 (3, 1H, 6.7 (d, 8.8 Hz, 2H, Har.); 7.13-7.25 (m, 22H, H ar.); 7.86 (d, 5.5 Hz, 1H, NH);

¹³C NMR (CDCl₃, 75.5 MHz)

68.8 (C6); 72.4 (C5); 73.7 (OCH₂Ph); 75.4 (OCH₂Ph); 75.8 (OCH₂Ph); 76.2 (OCH₂Ph); 78.3 (C2); 78.6 (C4); 83.3 (C3); 87.1 (d, 202 Hz, CFH); 98.0 (d, 20 Hz, CD; 116.2 (2Car.); 123.1 (2Car.); 127.9-129.1 (C ar.); 138.2; 138.5 (2C); 138.8 (C quat. ar.); 153.9 (Car.OH); 167.5 (d, 18 Hz, COCFH).

Mass (ESI+): 730.2 (M+23)

### Synthesis of Compounds 19b₁/19b₂ (FIG. 20)

To a suspension of acid 9b₁/9b₂ (253 mg; 0.42 mmol; 1.0 eq), peptide (240 mg; 0.42 mmol; 1.0 eq), HOBt (65 mg; 0.47 mmol; 1.1 eq), and NMM (143 mg; 1.41 mmol; 3.3 eq) in DMF (15 ml) under an argon atmosphere, EDCI (90 mg; 0.47 mmol; 1.1 eq) is added after 15 minutes. The reaction is stirred at room temperature for 4 days and then concentrated. A 1 N hydrochloric acid solution (10 ml) is added as well as dichloromethane, and the aqueous phase is extracted with DCM (3×20 ml). The organic phases are washed with a saturated NaCl solution (20 ml), dried on MgSO₄ and vacuum concentrated. The residue is purified by chromatography on a silica gel with as eluent a mixture of cyclohexane/AcOEt (1:1) to separate two diastereoisomers in the form of two white solids with a total yield of 64%.

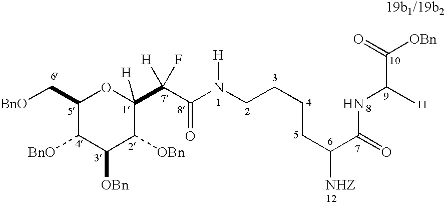

C₃₀H₆₆FN₃O₁₁ M=1024.21 g/mol

Characterization of 19b₁

Rf=0.52, eluent: cyclohexane/ethyl acetate (1:1)

¹⁹F NMR (CDCl₃, 282 MHz)

−197.6 (dd, ¹J_{F-H7′} 46.1, ²J_{F-H1′} 19.3)

¹H NMR (CDCl₃, 300 MHz) 1.26-1.68 (9H, m, H3, H4, H5, CH₃), 2.82-2.89 (1H, m, H2), 3.11-3.19 (1H, m, H2), 3.61 (1H, T, 3J 9.7), 3.71 (3H, sapp, 2H6′), 3.88-3.94 (2H, m), 4.10-4.15 (1H, m, H6), 4.49-4.63 (6H, m, H9), 4.78-5.24 (7H, m, H7′) 5.50 (1H, D, ³J_{H12-H6} 7.9, H12), 6.38 (1H, D, ³J_{H1-H}2 2.7, H1), 6.74 (1H, D, ³J_{H8-H}9 7.1, H8), 7.12-7.33 (30H, m, HAr),

¹³C NMR (CDCl₃, 75 MHz)

18.1 (C11), 22.3 (C4), 27.9 (C2), 32.1 (C5), 38.4 (C2), 48.3 (C9), 54.6 (C6), 67.0, 67.3, 69.1, 73.5, 74.6, 75.2, 75.6, 78.2, 77.4 (d, ³J_{C2′-F} 7.4, C2′), 79.0 (d, ²J_{C1′-F} 20.0, C1′), 79.4, 87.3, 90.8 (d, ¹J_{C7′-F} 191.3, C7′), 127.1, 127.3, 127.6, 127.7, 127.8, 127.8, 127.9, 128.0, 128.0, 128.1, 128.1, 128.2, 128.3, 128.5, 128.5, 128.6, 128.6, 128.7, 128.7, 135.4, 136.4, 138.1, 138.3, 138.4, 138.5, 156.3, 167.2 (d, ²J_{C8′-F} 19.4, C8′), 174.2, 172.6, 171.4.

Characterization of 19b₂

Rf=0.43, eluent: cyclohexane/ethyl acetate (1:1)

¹⁹F NMR (CDCl₃, 282 MHz)

−206.2 (dd, J_{F-H7}, 47.2, ²J_{F-H1} 29.0)

¹H NMR (CDCl₃, 300 MHz)

1.68-1.26 (9H, m, CH₃, 2H4, 2H5, 2H3), 3.28-3.11 (2H, m, 2H2), 3.73-3.62 (3H, m), 3.94-3.88 (2H, m), 4.65-4.46 (6H, m, H9), 5.24-4.72 (7H, m, HD, 5.73 (1H, D, 3J 7.6), 6.69 (1H, D, ³J6.9), 6.80 (1H, Sapp), 7.33-7.12 (30H, m, HAr),

Synthesis of Compound 20b₁ (FIG. 21)

In a flask, compound 19b₁ (30 mg; 0.028 mmol) is dissolved in tetrahydrofuran (5 ml) with a 1 N hydrochloric acid solution (1.2 eq) and palladium on carbon and placed under an atmosphere of hydrogen. The mixture is stirred for two days at room temperature. The reaction mixture is filtered and then concentrated. The crude product is taken up in dichloromethane (20 ml), which is eliminated, and then in water (10 ml), which is filtered. The aqueous phase is then concentrated thus leaving the desired product as a white solid with a yield of 77%.

Characterization of 20b₁

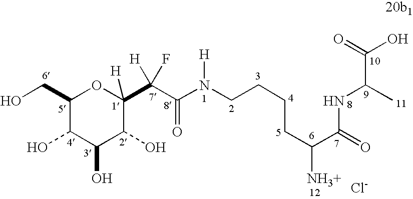

C₁₇H₃₀ClFN₃O₉ M=439.44 g/mol

¹⁹F NMR (D₂O, 282 MHz)

−209.9 (1F, dd, J_{F-H1′} 29.0, J_{F-H7′} 46.1)

¹H NMR (D₂O, 300 MHz)

1.34 (3H, D, 3, ³J_{H11-H9} 7.1, CH₃), 1.42-1.47 (2H, m, 2H4), 1.50-1.58 (2H, m, 2H2), 1.82-1.91 (2H, m, 2H5), 3.26-3.36 (4H, m, 2H2), 3.52-3.80 (3H, m, 2H6′, H2′), 3.95 (1H, T, ³J_{H6-H5} 6.5, H6), 4.15 (1H, Q, ³J_{H9-H11} 7.1, H9), 5.27 (1H, D, ²J_{H7′-F} 46.6, H7′)

¹³C NMR (D₂O, 75 MHz)

17.3 (C11), 21.6 (C4), 28.2 (C3), 30.7 (C5), 39.0 (C2), 53.4 (C6), 60.8 (C6′), 68.7 (d, ³J_{C2′}-F 5.1, C2′), 77.5, 69.7, 78.4 (d, ²J_{C1′-F} 18.1, C1′), 80.3, 88.5 (d, ¹J_{C7-F} 191.8, C7′, 169.2 (C10).

Mass spectrometry: ESI+: 440 (MH)+, 422 (MH−H₂0)+
Synthesis of Compound 20b₂ (FIG. 21)

In a flask, compound 19b₂ (35 mg; 0.032 mmol) is dissolved in tetrahydrofuran (10 ml) with a 1 N hydrochloric acid solution (1.2 eq) and palladium on carbon and placed under an atmosphere of hydrogen. The mixture is stirred for two days at room temperature. The reaction mixture is filtered and then concentrated. The crude product is taken up in dichloromethane (20 ml), which is eliminated, and then in water (10 ml), which is filtered. The aqueous phase is then concentrated thus leaving the desired product as a white solid with a yield of 75%.

Characterization of 20b₂

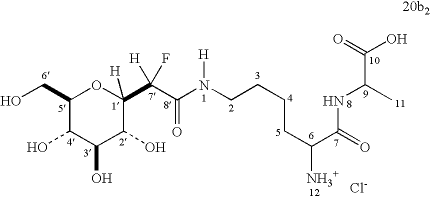

C₁₇H₃₀Cl FN₃O₉ M=439.44 g/mol

¹⁹F NMR (D₂O, 282 MHz)

−203.4 (1F, dd, J_{F-H1} ′ 25.8, J_{F-H7}, 48.3)

¹H NMR (D₂O, 300 MHz)

1.33 (3H, D, ³J_{H11-H9} 7.1, H11), 1.42-1.32 (2H, m, H4), 1.58-1.51 (2H, m, H3), 1.88-1.82 (2H, m, H5), 3.31-3.21 (2H, m, H2, 3.49-3.43 (2H, m), 3.64-3.58 (2H, m, H2′, H6′), 3.95-3.80 (3H, m, H6, H1′, H6′), 4.15 (1H, Q, ³J_{H9-H11} 7.1, H9), 5.20 (1H, D, ²J_{H7′-F} 47.7, H7′),

¹³C NMR (D₂O, 75 MHz)

17.2 (C11), 21.6 (C4), 28.1 (C3), 30.8 (C5), 38.8 (C2), 52.0 (C9), 53.4 (C6), 61.4 (C6′), 68.9 (d, ³J_{C2′-F} 7.0, C2′), 77.7, 69.9, 78.9 (d, ²J_{C1′-F} 19.1, C1′), 80.4, 90.6 (d, ¹J_{C7′-F} 190.9, C7′), 169.3 (C10).

Synthesis of Compound 21b₂ (FIG. 22)

In a flask under inert atmosphere containing aldehyde 10b₂ (130 mg; 0.216 mmol; 1 eq) in solution in anhydrous THF (4 ml), triethylphosphonoacetate (86 μl, 0.433 mmol, 2 eq), LiBr (38 mg, 0.433 mmol, 2 eq) and triethylamine (61 μl, 0.433 mmol, 2 eq) are added and the mixture is stirred for 12 hours. The mixture is hydrolyzed (20 ml water) and then extracted with ethyl acetate (3×15 ml). Next, the organic phases are recombined and washed with water (15 ml) and then a saturated NaCl solution (15 ml), and then dried on magnesium sulfate, filtered and then evaporated. The crude product is then purified by column chromatography on a silica gel with an eluent of cyclohexane/ethyl:acetate (80:20). The product is isolated with a 32% yield in the form of a colorless oil.

Characterization of 21b₂

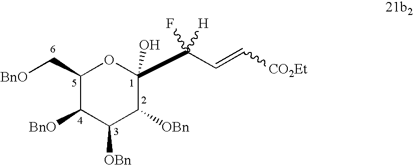

C₄₀H₄₃FO₈ M=670.76 g/mol

¹⁹F NMR (CDCl₃, 282.5 MHz)

−200.4 (dd, J_{F-H}=19.4 Hz and 47.3 Hz)

¹H NMR (CDCl₃, 300 MHz)

1.2 (t, 7.2 Hz, 3H, CH₃); 3.45 (d, 1H, OH); 3.5 (d, 9 Hz, 1H, H2); 3.65 (t, 9.4 Hz, 1H, H4); 3.60-3.75 (m, 2H, H6); 3.95 (m, 1H, H5); 4 (t, 10.5 Hz, 1H, H3); 4.07 (qdt, 7.2 Hz, 2H, CH₂); 4.47 (d, 12.3 Hz, 1H, OCH₂Ph); 4.55 (d, 12.2 Hz, 1H, OCH₂Ph); 4.58 (d, 10.7 Hz, 1H, OCH₂Ph); 4.60 (d, 11.1 Hz, 1H, OCH₂Ph); 4.76 (d, 11.5 Hz, 1H, OCH₂Ph); 4.8 (d, 11.5 Hz, 1H, OCH₂Ph); 4.83 (d, 10.1 Hz, 1H, OCH₂Ph); 4.90 (d, 10.9 Hz, 1H, OCH₂Ph); 4.97 (ddd, 1.7 Hz, 5.9 Hz and 46 Hz, 1H, CHF); 5.86 (dt, 1.7 Hz and 15.7 Hz, 1H, H); 6.76 (ddd, 4.8 Hz, 15.7 Hz and 20 Hz, 1H, CHF); 7.3 (m, 20H, Har.).

¹³C NMR (CDCl₃, 75.5 MHz)

14.5 (CH₃); 61.1 (CH₂); 68.7 (C6); 72.6 (C5); 73.8 (OCH₂Ph); 75.1 (OCH₂Ph); 75.4 (OCH₂Ph); 76.0 ((OCH₂Ph); 78.3 (C4); 78.5 (C2); 84.0 (C3); 92.7 (d, 181 Hz, CHF); 97.5 (d, 20 Hz, C1); 124.2 (d, 11.6 Hz, CH═); 128.0; 128.3; 128.6; 128.8 (2C); 128.9; 129.0 (C ar.); 137.7; 138.4; 138.7 (2C) (C quat. ar.), 165.8 (CO).

Synthesis of Compounds 22a₁/22a₂ and 23 (FIG. 23)

In a flask under inert atmosphere containing a mixture of acids 5a₁/5a₂ (120 mg; 0.195 mmol; 1 eq) and N-methylmorpholine (NMM) (64 μl; 65.6 mmol; 3 eq) in DMF (5 ml), EDCI (41 mg; 0.214 mmol; 1.1 eq) is added. Stirring is maintained for 24 hours and then the solvent is evaporated. The mixture is taken up in dichloromethane and washed twice with a 1 M HCl solution. The organic phase is dried on magnesium sulfate, filtered and concentrated. The crude product is then purified by column chromatography and the two isomers of the compounds are isolated with a 9/1 mixture of cyclohexane/ethyl acetate and a yield of 35%; the secondary compound is isolated with a mixture of cyclohexane/ethyl acetate and a yield of 10%.

Characterization of 22a₁/22a₂

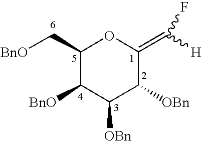

C₃₅H₅₅FO₅ M=554.65 g/mol

¹⁹F NMR (CDCl₃, 282.5 MHz)

−165.0 (d, 81 Hz, 1F)→41%

−159.1 (d, 77 Hz, 1F)→59%

¹H NMR (CDCl₃, 300 MHz)

3.63-3.76 (m, 3H, 2H6 and H3); 3.98 (td, 2.8 and 6 Hz, 1H, H5); 4.05 (t, 2.8 Hz, 1H, H4); 4.14 (dd, 1.8 and 7.6 Hz, 1H, H2); 4.40-4.81 (m, 8H, 40CH₂Ph); 6.43 (dd, 1.4 and 77 Hz, 1H, CHFmajo); 6.87 (d, 89 Hz, 1H, CHFmino); 7.23 (m, 20H, H ar.)

¹³C NMR (CDCl₃, 75.5 MHz)

68.5 (M) and 69.1 (m) (2C6); 73.2 (OCH₂Ph); 73.3 (OCH2Ph); 73.9 (OCH2Ph); 74.2 (C2 and C4); 74.3 (OCH2Ph); 79.1 (C5); 81.1 (C3); 128.0-128.9 (C ar.); 138.1; 138.4; 138.6 (2C) (C quat. ar.).

### Characterization of 23

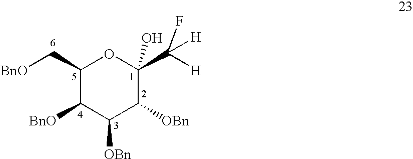

C₃₅H₃₇FO₆ M=572.66 g/mol

¹⁹F NMR (CDCl₃, 282.5 MHz)

−230.3 (t, 47 Hz, 1F)

¹H NMR (CDCl₃, 300 MHz)

3.63-3.73 (m, 4H of which 2H6); 3.90-3.96 (m, 2H); 4.2 (d, 47.3 Hz, 1H, CH₂F); 4.46 (d, 12.3 Hz, 1H, OCH₂Ph); 4.52 (d, 10.8 Hz, 1H, OCH₂Ph); 4.57 (d, 12.2 Hz, 1H, OCH₂Ph); 4.6 (d, 11 Hz, 1H, OCH₂Ph); 4.74 (d, 10.8 Hz, 1H, OCH₂Ph); 4.80 (d, 11.1 Hz, 1H, OCH₂Ph); 4.84 (d, 11.1 Hz, 1H, OCH₂Ph); 4.86 (d, 11 Hz, 1H, OCH₂Ph); 7.08-7.02 (m, 20H, H ar.).

¹³C NMR (CDCl₃, 75.5 MHz)

68.8 (C6); 72.5 (C5 or C2) 73.9 (OCH₂Ph); 75.3 (OCH₂Ph); 75.9 (OCH₂Ph); 76.1 (OCH₂Ph); 78.3 (C5 or C2); 78.6 (C4); 83.6 (C3); 83.9 (d, 179 Hz, CFH₂); 96.6 (d, 19 Hz, C1); 128.0-128.9 (C ar.); 137.8; 138.4; 138.6; 138.8 (C quat. ar.).

Synthesis of Compounds 24b₁/24b₂ (FIG. 24)

In a flask under inert atmosphere containing triphenylphosphine (230 mg; 0.88 mmol; 1.2 eq), tribromofluoromethane (CFBr₃) (86 μl; 0.88 mmol; 1.2 eq) and lactone 1b (394 mg; 0.731 mmol; 1 eq) in anhydrous THF, a solution of 1 M diethylzinc (Et₂Zn) in hexane or toluene (880 μl; 0.88 mmol, 1.2 eq) is slowly added dropwise. The mixture is stirred for 4 hours, then MeOH is added and the reaction mixture is concentrated. The crude product is then purified by column chromatography and the two isomers of the compounds are collected together with a 95/5 mixture of cyclohexane/ethyl acetate and a yield of 25%.

Characterization of 24b₁/24b₂

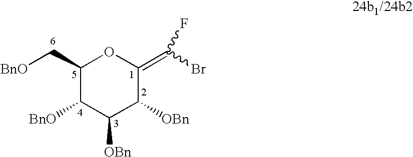

C₃₅H₃₄BrFO₅ M=633.54 g/mol

Rf=0.67, eluent: cyclohexane/ethyl acetate (8:2)

¹⁹F NMR (CDCl₃, 282.5 MHz)

−99.0 (s, 1F)

−118.6 (s, 1F)

¹H NMR (CDCl₃, 300 MHz)

3.63-3.7 (m, 3H including 2H6); 3.80-3.85 (m, 1H); 4.18 (td, 0.5H); 4.29-4.63 (m, 9.5H); 7.10-7.24 (m, 20H, H ar.)

### Test in the Presence of Glycosidase

To demonstrate the resistance of our compounds to glycosidases and thus to establish their stability, compound 17a₁ was reacted with galactosidases. Indeed, it is known that the following compounds undergo enzymatic break-down (FIG. 25).

The protocol used is as follows (FIG. 26)

A solution of compound 17a₁ (17.72 mg) in water (500 μl) is added to a solution of phosphate buffer (0.07 M; pH 7, 4 ml) containing α-galactosidase (5 units) and β-galactosidase (6.25 units) at 37° C. The reaction is monitored by ¹⁹F NMR. Samples are taken after 24, 48, 72, 96 and 120 hours. No change is observed and the starting product remains.

1. A C-glycoside compound of formula (I): 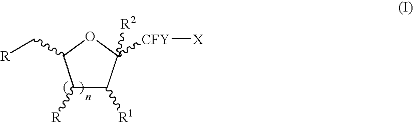
in which:
n is an integer equal to 1 or 2,
Y represents an atom of hydrogen, chlorine or bromine,
X is an atom of hydrogen or a linear or branched alkyl chain with at least one amine, amide, acid, ester, carbonyl, alcohol or aryl function or a carbonyl, ester, amide, amine or alcohol group,
R units are identical or different and represent an OH or OR′ group,
wherein R′ is a linear or branched alkyl, benzyl, benzoyl, acetyl, pivaloyl, trialkylsilyl, tertiobutyldiphenylsilyl group or one or more sugars,
R¹ represents OR′, NR″R″′, N₃, or a phthalimide,
R″ and R″′, identical or different, represent an atom of hydrogen or a linear or branched alkyl, aryl, benzyl, benzoyl, acetyl, alkyloxycarbonyl, allyloxycarbonyl or benzyloxycarbonyl group,
R² represents an atom of hydrogen, a halogen or an OH, OR′, NR″R″′ or N₃ group,
as well as derivatives of same in form of a base, a mineral or organic acid addition salt, a hydrate or a physiologically or pharmaceutically acceptable solvate: with the exception of the following compounds:
methyl 3,4,6,tri-O-benzoyl-1-deoxy-1-fluoro-β-D-fructofuranoside,
2-deoxy-2-fluoro-4,5,7-tris-O-(phenylmethyl)-D-arabino-3-heptulofuranosonic acid ethyl ester,
2-deoxy-2-fluoro-4,5,7-tris-O-(phenylmethyl)-D-arabino-3-heptulofuranosonic acid
1-deoxy-1-fluoro-3,4,6-tris-O-(phenylmethyl)-D-fructofuranose
1-deoxy-1-fluoro-3,4-bis-O-(phenylmethyl)-D-fructofuranose diacetate, and
1-deoxy-1-fluoro-3,4-bis-O-(phenylmethyl)-D-fructofuranose.

2. The compound according to claim 1, wherein the linear or branched alkyl groups are groups with 1 to 15 carbon atoms.

3. A method for preparing a compound of formula (I) according to claim 1 in which Y represents a hydrogen molecule, wherein it comprises a Reformatsky addition reaction of an alkyl bromofluoroacetate in the presence of zinc with the lactones of formula (II): 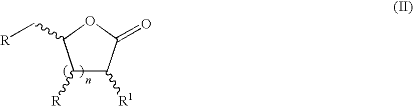
with n, R and R¹ as defined in claim 1.

4. A method for producing a compound of formula (I) according to claim 1 in which X and Y represent hydrogen atoms and R² represents an OH group, wherein a compound of formula (I) as defined in claim 1 in which R²═OH, Y═H and X═CO₂H is reacted with a peptide coupling agent in the presence of a tertiary amine.

5. A method for producing a compound of formula (I) according to claim 1 in which Y represents a hydrogen atom, wherein it comprises a reaction of an alkyl dibromofluoroacetate in the presence of diethylzinc and triphenylphosphine with the lactones of formula (II) as defined in claim 3.

6. A method for producing a compound of formula (I) according to claim 1 in which Y represents a halogen atom wherein it comprises a reaction of alkyl dihalofluoroacetate in the presence of diethylzinc with the lactones of formula (II) as described in claim 3.

7. A method according to one of the claims to 3 to 6, wherein the lactones of formula (II) are obtained by steps of protection by benzylation of sugar, followed by acid hydrolysis of the anomeric position and then its oxidation.

8. A method for preparing a compound of formula (I) according to claim 1 in which R² represents a chlorine or bromine atom, wherein a compound of formula (I) as defined in claims 1 in which R²═OH is halogenated.

9. A compound of formula (III): 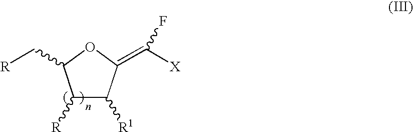
with n, R, R¹ and X as defined in claim 1.

10. A method for preparing a compound formula (III) according to claim 9 in which X═Br, wherein it comprises a reaction of a lactone of formula (II) as defined in claim 3 in the presence of tribromofluoromethane, triphenylphosphine and diethylzinc.

11. A method for preparing a compound of the general formula (I) according to claim 1 wherein R²═H and Y═H by the reduction of the double bond of the compound of general formula (III) as defined in claim 9.

12. A compound according to claim 1, wherein it is of following formula (IV): 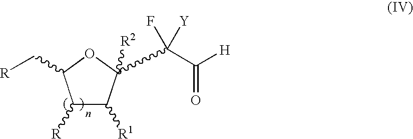
in which:
n is an integer equal to 2,
Y represents a hydrogen atom,
R² represents a hydrogen atom or an OH or OR′ group and
R¹ is as defined in claim 1.

13. A compound of following formula (V): 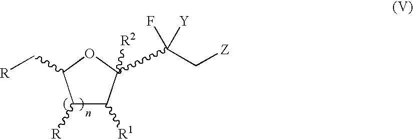
in which:
n is an integer equal to 2,
Y represents a hydrogen atom,
represents a hydrogen atom or an OH or OR′ group,
Z represents OH or OR³ with R³=alkyl, benzyl, mesyl, tosyl, triflate or a halogen and
R¹ is as defined in claim 1.

14. A compound of following formula (VI): 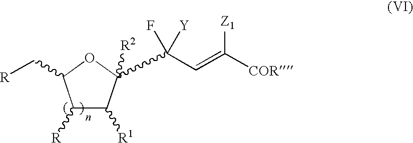
in which:
n is an integer equal to 2,
Y represents a hydrogen atom,
R² represents a hydrogen atom or an OH or OR′ group,
Z₁ represents H or NR″R″′ with R″ and R″′, identical or different, representing a hydrogen atom or a linear or branched alkyl, aryl, benzyl, benzoyl, acetyl, alkyloxycarbonyl, allyloxycarbonyl or benzyloxycarbonyl group,
R″″ represents OR″ or NR″R″′ or an amino acid with R″ and R″′ as defined above and
R¹ is as defined in claim 1.

15. A compound of following formula (VII): 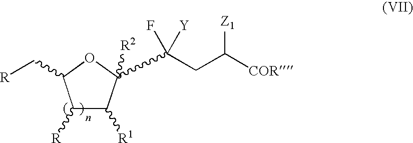
in which:
n is an integer equal to 2,
Y represents an atom of hydrogen,
R² represents an atom of hydrogen or an OH or OR′ group,
Z₁ represents H or NR″R″′ with R″ and R″′, identical or different, representing an atom of hydrogen or a linear or branched alkyl, aryl, benzyl, benzoyl, acetyl, alkyloxycarbonyl, allyloxycarbonyl or benzyloxycarbonyl group,
R″″ represents OR″ or NR″R″′ or an amino acid with R″ and R″′ as defined above and
R¹ is as defined in claim 1.

16. A compound according following formula (VIII): 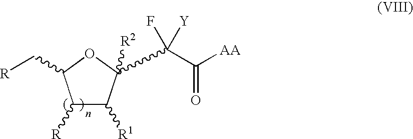
in which:
n is an integer equal to 2,
Y represents an atom of hydrogen,
R² represents an atom of hydrogen or an OH or OR′ group,
AA represents an amino acid or peptide and
R¹ is as defined in claim 1.

17. A compound of following formula (IX): 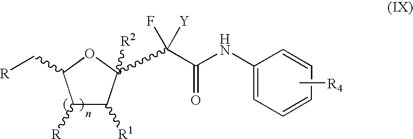
in which:
n is an integer equal to 2,
Y represents an atom of hydrogen,
R² represents an atom of hydrogen or an OH or OR′ group,
R⁴ represents a hydrogen, halogen, NR″R″′, OH or OR″, with R″ and R″′, identical or different, representing an atom of hydrogen or a linear or branched alkyl, aryl, benzyl, benzoyl, acetyl, alkyloxycarbonyl, allyloxycarbonyl or benzyloxycarbonyl group and
R¹ is as defined in claim 1.

18. A compound of formula (I) according to claim 1, in which R² consists represents an OH group wherein it is present in open forms of sugar when it is solution in polar and protic solvents.

19. A method for preparing a compound of formula (I) according to claim 1 in which R² represents a hydrogen atom, wherein a compound of formula (I) as defined in claim 1 in which R²═Cl or Br is reduced.

20. A method for preparing a compound of formula (III) according to claim 9 in which X═H by reacting a compound of formula (I) according to claim 1 in which X═CO₂H, R²═OH and Y═H in the presence of a peptide coupling agent, and in the presence of a tertiary amine.

21. A compound according to one of the claims 1, 9 or 12 to 17, wherein it is chosen among:

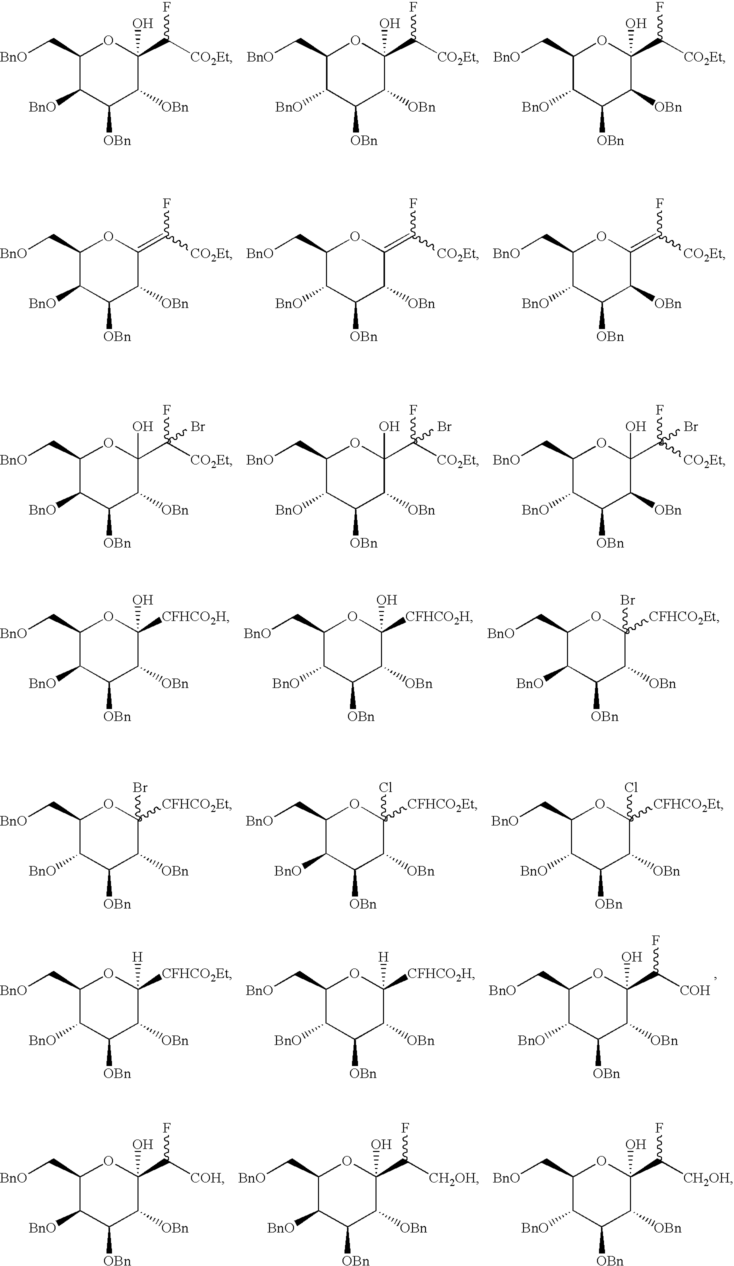

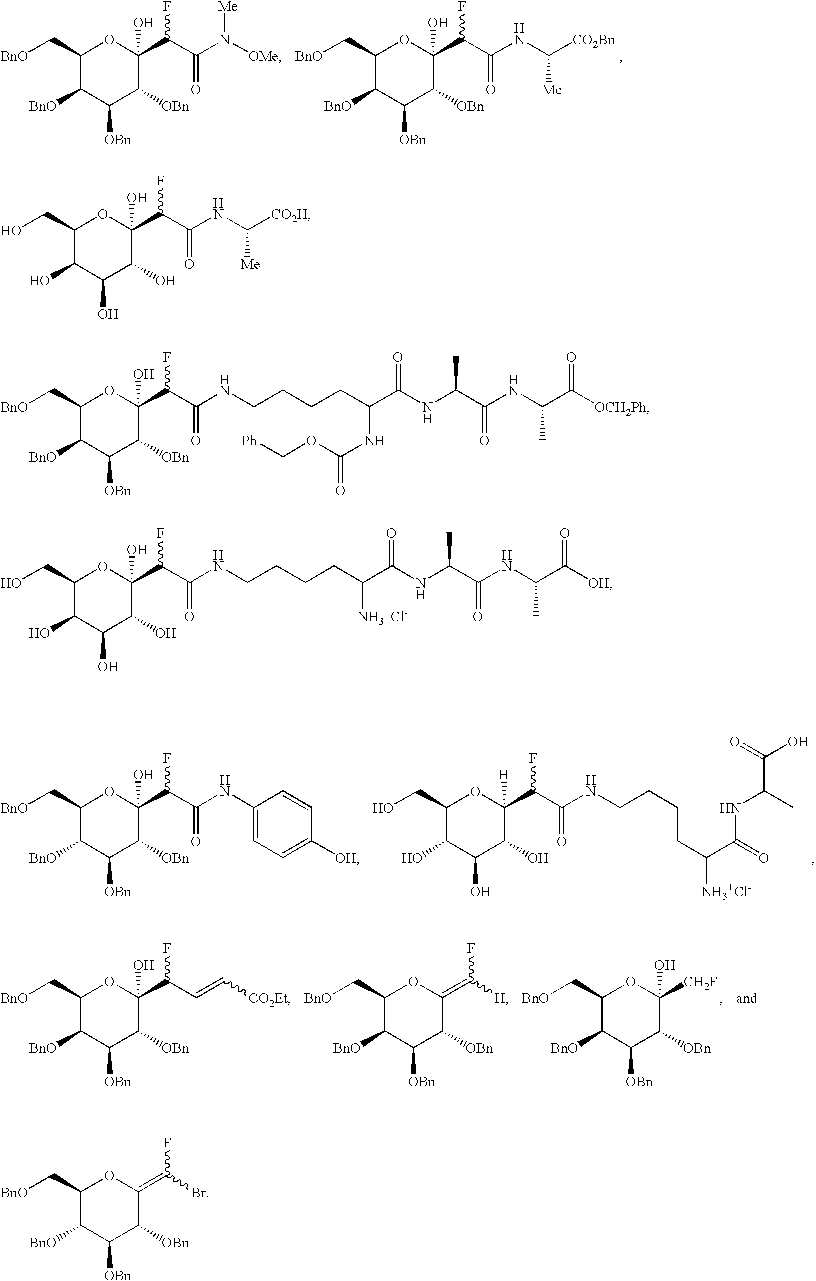

22. The method according to claim 4 or 20, wherein the tertiary amine is N-methylmorpholine or diisopropylamine.

23. The method according to claim 4 or 20, wherein the peptide coupling agent is 3-ethyl-1(N,N-dimethylaminopropylcarbodiimide or dicyclohexyl carbodiimide.

24. The method according to claim 6, wherein Y represents bromine or chlorine.

25. The compound of formula (I) according to claim 18, wherein the open forms of sugar are furanose and pyranose.
